(19) United States
(12) Patent Application Publication
(10) Pub. No.: US 2015/0274703 A1
(43) Pub. Date: Oct. 1, 2015

(54) 1H-Indole-3-Carboxamide Derivatives And Use Thereof As P2Y12 Antagonists

(71) Applicant: SANOFI, Paris (FR)

(72) Inventors: Christophe Boldron, Paris (FR); Alain Badorc, Paris (FR); Angelina Besse, Paris (FR); Valérie Fossey, Paris (FR); Gernot Zech, Frankfurt am Main (DE)

(21) Appl. No.: 14/438,337

(22) PCT Filed: Oct. 24, 2013
(86) PCT No.: PCT/EP2013/072322

§ 371 Date: Apr. 24, 2015

(30) Foreign Application Priority Data
Oct. 26, 2012 (EP) 12306334.9

(51) Int. Cl. C07D 403/12; C07D 401/14

(52) U.S. Cl. CPC C07D 403/12; C07D 401/14

(57) ABSTRACT

The invention relates to compounds corresponding to formula (I):

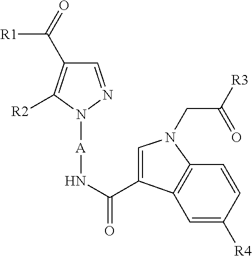

and to the use thereof as P2Y 12 receptor antagonists for the treatment of cardiovascular diseases.

The present invention relates to novel derivatives of N-[(1H-pyrazol-1-yl)aryl]-1H-indole or 1H-indazole-3-carboxamide, to the preparation thereof and to the therapeutic use thereof.

The compounds according to the present invention are reversible antagonists of the P2Y12 purinergic receptor. These compounds are platelet aggregation inhibitors which exert a powerful antithrombotic effect. They can be used for the treatment and prophylaxis of cardiovascular disorders, such as thromboembolic diseases and restenoses.

In the industrialized world, medical complications associated with the occurrence of thrombosis represent one of the main causes of mortality. Some examples of pathological conditions associated with the development of thrombosis include acute myocardial infarction, unstable angina (pectoris) and chronic stable angina, transient ischemic attacks, strokes, peripheral vascular disease, pre-eclampsia and eclampsia, deep vein thrombosis, embolisms (cerebral, pulmonary, coronary artery, renal, etc.), disseminated intravascular coagulation, or thrombocytopenic thrombotic purpura. Risks of thrombotic and restenotic complications also exist during and following invasive surgical procedures, such as angioplasty, carotid endarterectomy, aortocoronary bypass graft, or else the implantation of stents or endovascular prostheses.

Arterial thromboses can occur following a vessel wall lesion or rupture of an atherosclerosis plaque. Platelets play an essential role in the formation of these thromboses. Platelets can be activated either by mediators released into the bloodstream by circulating cells or by damaged endothelial cells present along the vessel wall, or by thrombogenic molecules of the subendothelial matrix—such as collagen—exposed during vascular lesions. In addition, platelets can also be activated under conditions of blood flow at high shear stress which are found in stenotic vessels. After activation, circulating platelets adhere and accumulate at the vascular lesion so as to form a thrombus. During this process, the thrombi generated can be sufficiently voluminous to partially or totally block the blood flow in the vessel.

In the veins, thrombi can also form where there is stasis or slow blood flow. By virtue of their nature, these venous thrombi can produce emboli which move through the vascular system. These emboli are then capable of blocking the blood flow in vessels that are more remote, such as the pulmonary or coronary arteries.

Numerous studies have demonstrated that adenosine-5′-diphosphate (ADP) is an essential mediator of platelet activation and aggregation, playing a key role in the initiation and progression of thrombus formation (Maffrand et al, Thromb. Haemostas. (1988) 59, 225-230; Herbert et al, Arterioscl. Thromb. (1993) 13, 1171-1179).

ADP is released into the circulation by damaged red blood cells and endothelial cells of the atherosclerotic wall, and more specifically secreted by activated platelets where ADP is stored in the dense granules at very high concentration. ADP-induced platelet aggregation is triggered by the binding of said ADP to two specific purinergic receptors, expressed on the cell membrane of human platelets: P2Y1 and P2Y12. The P2Y1 receptor, coupled with stimulation of PLCβ via Gaq, is responsible for the mobilization of internal calcium stores, the change in shape of the platelets, and transient aggregation induced by ADP. The P2Y12 receptor, coupled with inhibition of adenylcyclase via Gαi2 and with activation of PI-3 kinase, is responsible for amplification of the responses and stabilization of aggregation (Gachet, Thromb. Haemost. (2001) 86, 222-232; Andre et al, J. Clin. Invest. (2003) 112, 398-406). The use of P2Y1−/− transgenic mice (Gachet et al, J Clin invest (1999) 104, 1731-1737; Gachet et al, Circulation (2001) 103, 718-723; Gachet et al, Haematologia (2002) 87, 23) and P2Y12−/− transgenic mice (Conley et al, Nature (2001) 409, 202-207) has made it possible to demonstrate the importance of these two receptors in the development of thromboses in vivo. In humans, genetic defects for P2Y12 have been described as being associated with a hemorrhagic phenotype and with a pronounced impairment of ADP-induced platelet aggregation (Kanakura et al, J Thromb Haemost. (2005) 3, 2315-2323).

The use of Clopidogrel in human clinical practice has provided proof that blocking the P2Y12 receptor with an antagonist represents a key therapeutic strategy in the treatment of cardiovascular diseases. Clopidogrel is a prodrug of the thienopyridine family, the active metabolite of which binds covalently to the P2Y12 receptor, resulting in irreversible inhibition of platelet activity in vivo. (Savi et al, Biochem Biophys Res Commun (2001) 283, 379-383; Savi et al, Proc Natl Acad Sci (2006) 103, 11069-11074). This molecule had initially shown its efficacy in several clinical trials, reducing the risk of cardiovascular events in patients at risk (“A randomised, blinded, trial of clopidogrel versus aspirin in patients at risk of ischaemic events (CAPRIE)” CAPRIE steering committee Lancet (1996) 348, 1329-1339; “The Clopidogrel in Unstable Angina to Prevent Recurrent Events (CURE). Effects of Clopidogrel in Addition to Aspirin in Patients with Acute Coronary Syndromes without ST-Segment Elevation” CURE steering committee N Engl J Med (2001) 345, 7, 494-502).

Synthetic antagonists of the P2Y12 receptor which exhibit antiplatelet and antithrombotic activity have been described. Nevertheless, the need for new antagonists which have superior properties remains, in particular the need for reversible antagonists with a better benefit/risk ratio.

The present invention relates to compounds corresponding to formula (I):

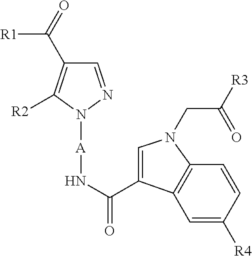

in which:
A represents a divalent radical chosen from:

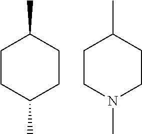

R₁ represents a (C₁-C₄)alkyl;
R₂ represents a (C₁-C₃)alkyl;
R₃ represents an —NR₇R₈ group, R₇ and R₈, together with the nitrogen atom to which they are bonded, constitute a saturated heterocycle comprising from 4 to 6 ring members and which may contain another nitrogen atom; said heterocycle being substituted with at least one (C₁-C₃)alkyl which is unsubstituted or substituted with at least one of the following groups:
   one, two or three halogen atoms, or
   a (C₁-C₃)alkoxy which is unsubstituted or substituted with one, two or three halogen atoms;
R₄ represents a halogen atom.

The compounds of formula (I) can comprise one or more asymmetric carbon atoms. They can therefore exist in the form of enantiomers or diastereoisomers. These enantiomers, diastereoisomers, and also mixtures thereof, including racemic mixtures, form part of the invention.

The compounds of formula (I) may exist in the form of bases or salified with acids or bases, in particular pharmaceutically acceptable acids or bases. Such addition salts form part of the invention.

These salts are advantageously prepared with pharmaceutically acceptable acids or bases, but the salts of other acids or bases which are of use, for example, for purifying or isolating the compounds of formula (I) are also part of the invention.

The term “halogen atom” is intended to mean a bromine, chlorine, fluorine or iodine atom.

The term “alkyl” is intended to mean a linear or branched alkyl radical having from one to four carbon atoms, such as the methyl, ethyl, propyl, isopropyl, butyl, isobutyl, sec-butyl or tert-butyl radical.

The term “alkoxy” is intended to mean a linear or branched alkoxy radical having from one to three carbon atoms, such as the methoxy, ethoxy, propoxy, isopropoxy or butoxy radical.

The term “heterocycle” is intended to mean a cyclic alkyl group comprising from 4 to 6 atoms forming this ring, and one or two of which are selected heteroatoms. Mention may in particular be made of pyrrolidine, imidazoline, piperidine, pyrazolidine and piperazine groups.

Among the compounds of formula (I) according to the invention, mention may be made of a subgroup of compounds in which A represents:

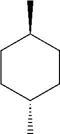

Another subgroup of compounds of formula (I) is such that A represents:

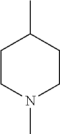

Another subgroup of compounds of formula (I) is such that R₁ represents an n-propyl group.

Another subgroup of compounds of formula (I) is such that R₃ represents a piperazine group substituted with at least one (C₁-C₃)alkyl which is unsubstituted or substituted with at least one of the following groups:
one, two or three halogen atoms, or
a (C₁-C₃)alkoxy which is unsubstituted or substituted with one, two or three halogen atoms.

Another subgroup of compounds of formula (I) is such that R₄ represents a chlorine atom.

The subgroups above, taken separately or in combination, also form part of the invention.

Among the compounds of formula (I) that are subjects of the invention, mention may be made in particular of the following compounds:
5-Chloro-1-[2-(4-methylpiperazin-1-yl)-2-oxoethyl]-1H-indole-3-carboxylic acid [4-(4-butyryl-5-methylpyrazol-1 -yl)cyclohexyl]amide
5-Chloro-1-[2-(4-methylpiperazin-1-yl)-2-oxoethyl]-1H-indole-3-carboxylic acid [4-(4-butyryl-5-methylpyrazol-1-yl)piperidin-1-yl]amide
5-Chloro-1-{2-oxo-2-[4-(3,3,3-trifluoropropyl)piperazin-1-yl]ethyl}-1H-indole-3-carboxylic acid [4-(4-butyryl-5-methylpyrazol-1-yl)cyclohexyl]amide
5-Chloro-1-{2-[4-(2-methoxyethyl)piperazin-1-yl]-2-oxoethyl}-1 -1H-indole-3-carboxylic acid [4-(4-butyryl-5-methylpyrazol-1-yl)cyclohexyl]amide
5-Chloro-1-{2-[4-(2-methoxyethyl)piperazin-1-yl]-2-oxoethyl}-1-1H-indole-3-carboxylic acid [4-(4-butyryl-5-methylpyrazol-1-yl)piperidin-1-yl]amide
5-Chloro-1-{2-oxo-2-[4-(2-trifluoromethoxyethyl)piperazin-1-yl]ethyl}-1H-indole-3-carboxylic acid [4-(4-butyryl-5 -methylpyrazol-1-yl)piperidin-1-yl]amide

in the form of a base or of an addition salt with an acid or with a base.

It should be noted that the above compounds were named according to the IUPAC nomenclature by means of the AutoNom software (Beilstein Informations system).

In the text hereinbelow, the term “protecting group Pg” is intended to mean a group that can, firstly, protect a reactive function such as a hydroxyl or an amine during a synthesis and, secondly, regenerate the intact reactive function at the end of the synthesis. Examples of protecting groups and also protection and deprotection methods are given in “Protective Groups in Organic Synthesis”, Greene et al., 4th Edition John Wiley & Sons, Inc., New York, 2007.

In the text hereinbelow, the term “leaving group” is intended to mean a group which can be easily cleaved from a molecule by breaking a heterolytic bond, with the departure of a pair of electrons. This group may thus be readily replaced with another group, for example during a substitution reaction. Such leaving groups are, for example, halogens or an activated hydroxyl group such as a methanesulfonate, benzenesulfonate, p-toluenesulfonate, triflate, acetate, etc. Examples of leaving groups and also references for their preparation are given in “Advanced Organic Chemistry”, M. B. Smith and J. March, 6th Edition, Wiley Interscience, 2007, p. 496-501.

In accordance with the invention, the compounds of formula (I) can be prepared according to a process which is characterized in that:

a compound of formula (IIB):

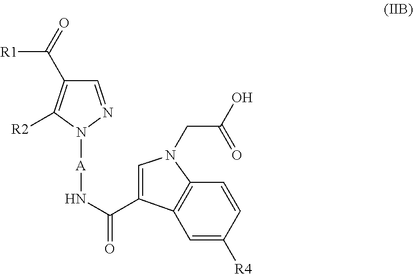

in which A, R1, R2 and R4 are as defined for a compound of formula (I), is reacted with an amine of formula (III):

HR₃ (III)

in which R₃ is as defined for a compound of formula (I).

Optionally, the compound of formula (I) is converted into a salt thereof with inorganic or organic acids.

The reaction is carried out in a presence of a base, for instance triethylamine, 4-dimethylaminopyridine, N,N-diisopropylethylamine, N-methylmorpholine, N-ethylmorpholine or pyridine and in the presence of a coupling agent, for instance bis(2-oxo-1,3-oxazolidin-3-yl)phosphinic chloride, isobutyl chloroformate, 1,1′-carbonyldiimidazole, 1-(3-dimethylaminopropyl)-3-ethylcarbodiimide hydrochloride, N-hydroxybenzotriazole, or ethyl 2-cyano-2-(hydroxyimino)acetate. The solvent used is, for example, DCM, 1,2-dichloroethane or N,N-dimethylformamide. The temperature of the reaction is between -10° C. and the reflux temperature of the solvent.

The compounds of formula (I) can also be prepared by reacting an acid or an activated functional derivative of this acid of formula:

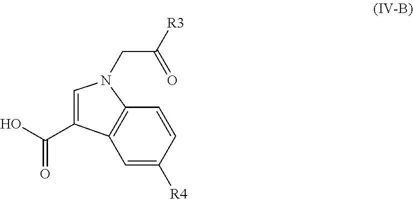

in which R3 and R4 are as defined for a compound of formula (I), with a compound of formula:

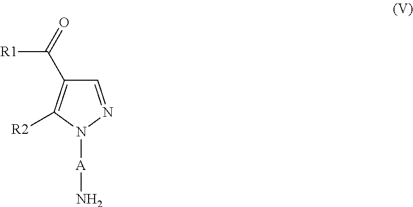

in which A, R1 and R2 are as defined for a compound of formula (I).

When a compound of formula (V) is treated with the acid of formula (IV-B) itself, the process is carried out in the presence of a coupling agent used in peptide chemistry, such as 1,3 -dicyclohexylcarbodiimide or 1-ethyl-3-(3-dimethylaminopropyl)carbodiimide hydrochloride or benzotriazol-1-yl-oxytris(dimethylamino)phosphonium hexafluorophosphate or N-hydroxybenzotriazole or benzotriazol-1-yl-oxytris(pyrrolidino)phosphonium hexafluorophosphate or 2-(1H-benzotriazol-1-yl)-1,1,3,3-tetramethyluronium tetrafluoroborate or 1-(3-dimethylaminopropyl)-3-ethylcarbodiimide hydrochloride, in the presence of a base such as triethylamine, N,N-diisopropylethylamine or 4-dimethylaminopyridine, in a solvent such as DCM, 1,2-dichloroethane, N-N-dimethylformamide or tetrahydrofuran at a temperature of between −10° C. and the reflux temperature of the solvent.

As activated functional derivative of the acid (IV-B), use may be made of the acid chloride, the anhydride, a mixed anhydride, a C₁-C₄ alkyl ester in which the alkyl is linear or branched, or an activated ester, for example the p-nitrophenyl ester.

Thus, it is possible, for example, to react the acid chloride obtained by reacting thionyl chloride or oxalyl chloride with the acid of formula (IV-B), with the compound of formula (V) in a solvent, such as a chlorinated solvent (DCM, 1,2-dichloroethane, chloroform for example), an ether (tetrahydrofuran, dioxane for example), an amide (N,N-dimethylformamide for example) or pyridine, under an inert atmosphere, at a temperature of between 0° C. and the reflux temperature of the solvent, in the presence of a tertiary amine such as triethylamine, N-methylmorpholine, pyridine, 4-dimethylaminopyridine or 1,8-diazabicyclo[5.4.0]undec-7-ene.

In particular, certain compounds of formula (I) may be prepared from other compounds of formula (I).

The compounds of formula (I) thus obtained may be subsequently separated from the reaction medium and purified according to standard methods, for example by crystallization or silica column chromatography.

The compounds of formula (I) thus obtained are isolated in the form of a free base or of a salt, according to the standard techniques.

The compounds of formula (IIB) can be prepared according to a process in which a compound of formula (IIA):

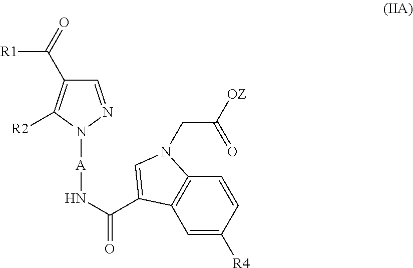

in which A, R1, R2 and R4 are as defined for a compound of formula (I) and Z represents a (C₁-C₄) alkyl, is hydrolyzed in an acidic or basic medium.

Optionally, the compound of formula (IIA) is converted into a salt thereof with inorganic or organic bases.

The reaction is carried out in an acidic medium through the action of a strong acid, for instance hydrochloric acid or sulfuric acid, in a solvent, in particular a polar solvent, such as, for example, dioxane or water, and at a temperature of between −10° C. and 110° C.

The reaction is carried out in a basic medium through the action of an alkaline base, for instance potassium hydroxide, lithium hydroxide or sodium hydroxide, in a solvent, in particular a polar solvent, such as, for example, dioxane, tetrahydrofuran, water, methanol, ethanol, or a mixture of these solvents, and at a temperature of between −10° C. and the reflux temperature of the solvent.

The compounds of formula (IIA) can be prepared by reacting an acid or an activated functional derivative of this acid of formula:

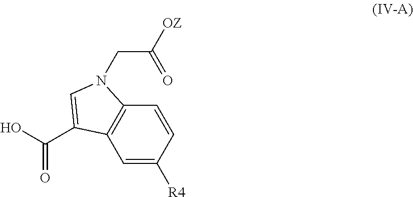

in which R4 is as defined for a compound of formula (I) and Z represents a (C₁-C₄) alkyl, with a compound of formula:

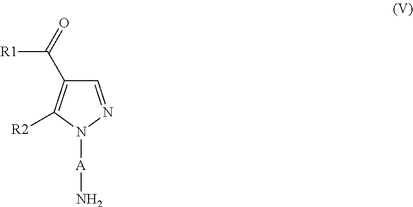

in which A, R1 and R2 are as defined for a compound of formula (I).

When a compound of formula (V) is treated with the acid of formula (IV-A) itself, the process is carried out in the presence of a coupling agent used in peptide chemistry, such as 1,3-dicyclohexylcarbodiimide or 1-ethyl-3-(3-dimethylaminopropyl)carbodiimide hydrochloride or benzotriazol-1-yl-oxytris(dimethylamino)phosphonium hexafluorophosphate or benzotriazol-1-yl-oxytris(pyrrolidino)phosphonium hexafluorophosphate or 2-(1H-benzotriazol-1-yl)-1,1,3,3 -tetramethyluronium tetrafluoroborate or 1-(3-dimethylaminopropyl)-3-ethylcarbodiimide hydrochloride, in the presence of a base such as triethylamine, N,N-diisopropylethylamine or 4-dimethylaminopyridine, in a solvent such as DCM, 1,2-dichloroethane, N-N-dimethylformamide or tetrahydrofuran at a temperature of between −10° C. and the reflux temperature of the solvent.

As activated functional derivative of the acid (IV-A), use may be made of the acid chloride, the anhydride, a mixed anhydride, a C₁-C₄ alkyl ester in which the alkyl is linear or branched, or an activated ester, for example the p-nitrophenyl ester.

Thus, it is possible, for example, to react the acid chloride obtained by reacting thionyl chloride or oxalyl chloride with the acid of formula (IV-A), with the compound of formula (V) in a solvent, such as a chlorinated solvent (DCM, 1,2-dichloroethane, chloroform for example), an ether (tetrahydrofuran, dioxane for example), an amide (N,N-dimethylformamide for example) or pyridine, under an inert atmosphere, at a temperature of between 0° C. and the reflux temperature of the solvent, in the presence of a tertiary amine such as triethylamine, N-methylmorpholine, pyridine, 4-dimethylaminopyridine or 1,8-diazabicyclo[5.4.0]undec-7-ene.

The compounds of formula (III) may be known, commercially available or prepared according to methods known to those skilled in the art.

The compounds of formula (V) are prepared by means of a reaction for deprotection of a compound of formula:

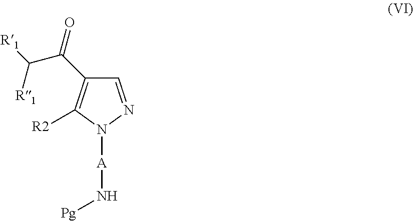

in which A and R2 are as defined for a compound of formula (I), R′₁ represents a hydrogen atom or a (C₁-C₃)alkyl, R″₁ represents a hydrogen atom or a —COOZ group, Z represents a (C₁-C₄) alkyl and Pg represents a protecting group, preferably a tert-butoxycarbonyl or benzyloxycarbonyl group.

The reaction is carried out through the action of an acid, for instance hydrochloric acid or trifluoroacetic acid, optionally in the presence of a solvent, for example water or dioxane, and at a temperature between ambient temperature and the reflux temperature of the reaction medium.

The compounds of formula (VI) for which R″₁ ═H are prepared by reacting a compound of formula:

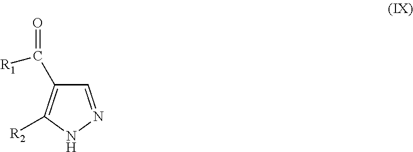

in which R₁ and R₂ are as defined for a compound of formula (I), with a compound of formula:

Y-A-NHPg (X)

in which A is as defined for a compound of formula (I), Y represents a leaving group such as a halogen atom, a methanesulfonate, a benzenesulfonate, a p-toluenesulfonate, a triflate or an acetate and Pg represents a protecting group, preferably a tert-butoxycarbonyl or benzyloxycarbonyl group.

The reaction is carried out in the presence of a base, for instance potassium carbonate, cesium carbonate or potassium tert-butoxide. The reaction is carried out in a solvent, for instance dimethyl sulfoxide, N,N-dimethylformamide, dioxane or tetrahydrofuran and at a temperature of between 0° C. and 150° C.

The compounds of formula (VI) for which R″₁═COOZ can be prepared according to SCHEME I hereinafter, in which R2 is as defined for a compound of formula (I), Z represents a (C₁-C₄) alkyl and Pg represents a protecting group, preferably a tert-butoxycarbonyl or benzyloxycarbonyl group.

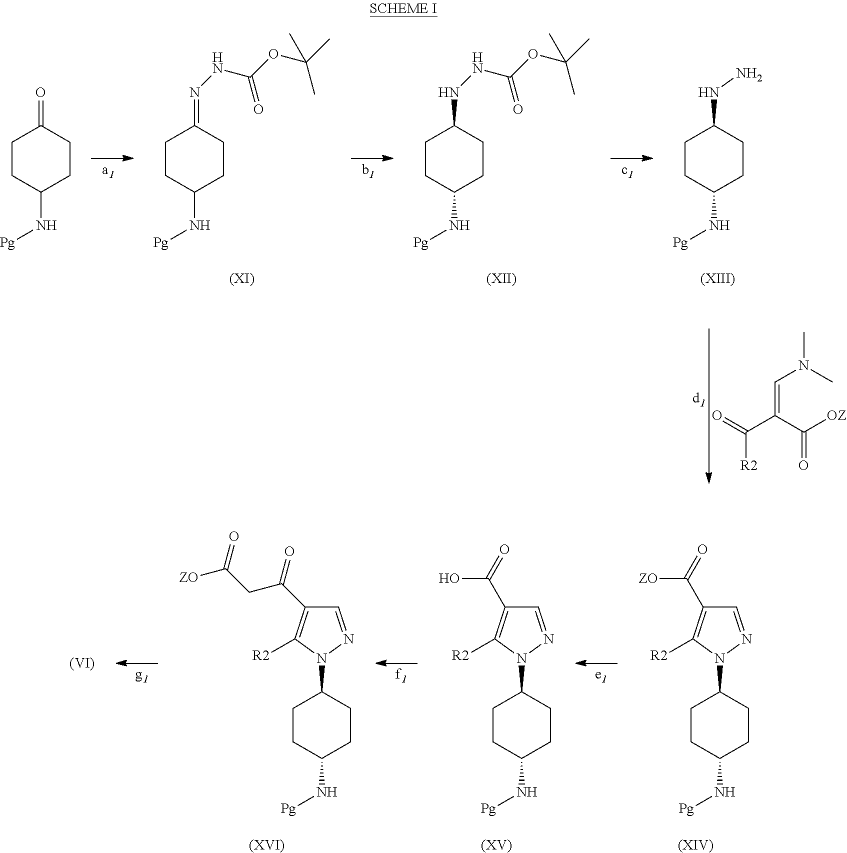

In step a₁ of SCHEME I, a protected 4-aminocyclohexanone is condensed with tert-butyl carbazate so as to obtain the compound (XI).

In step b₁, the compound (XI) is reduced using sodium cyanoborohydride in methanol, then the isomer of trans configuration (XII) is separated by crystallization from ethyl acetate in the presence of a stoichiometric amount of hydrochloric acid.

In step c₁, the hydrazine function is deprotected in the presence of excess hydrochloric acid in dioxane so as to obtain the compound (XIII).

In step d₁, the hydrazine derivative (XIII) is condensed with 2-[1-dimethylaminomethylidene]-3-oxobutyric acid ethyl ester in the presence of triethylamine in ethanol at reflux in order to generate the pyrazole derivative (XIV).

In step e₁, the compound of formula (XIV) is hydrolyzed in the presence of aqueous sodium hydroxide in a mixture of tetrahydrofuran and ethanol so as to obtain the corresponding carboxylic acid (XV).

In step f₁, the compound of formula (XV) is reacted with 1,1′-carbonyldiimidazole and then, without isolating it, the resulting intermediate compound is treated with a magnesium salt of a hemi-ester of malonic acid according to the method described in Angew. Chem. Int. Ed. Engl (1979) 18(1), 72-74.

In step g₁, the resulting compound of formula (XVI) is alkylated by reacting a (C₁-C₃)alkyl halide, mesylate or tosylate, in the presence of a base such as, for example, potassium carbonate, and of a phase-transfer catalyst such as, for example, tetrabutylammonium bromide in a solvent, for instance tetrahydrofuran, and at a temperature between 0° C. and the reflux temperature of the solvent.

The compounds of formula (XI) in which R₁═(C₁-C₄)alkyl can be prepared according to SCHEME II hereinafter, in which R2 is as defined for a compound of formula (I) and Pg represents a protecting group, preferably a trityl group.

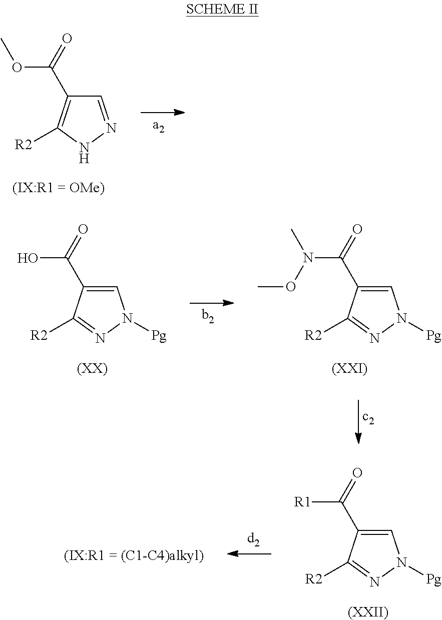

In step a₂ of SCHEME II, the nitrogen atom of the compound of formula (IX) for which R₁═OMe is protected, in particular with a trityl group, then the intermediate compound obtained is hydrolyzed in a basic medium.

In step b₂, the resulting compound of formula (XX) is reacted with N-methoxymethanamine, in the presence of a coupling agent such as, for example, bis(2-oxo-1,3-oxazolidin-3-yl)phosphinic chloride and in the presence of a base such as, for example, 4-dimethylaminopyridine. The reaction is carried out in a solvent, for instance dichloromethane, and at a temperature of between 0° C. and ambient temperature.

The resulting compound of formula (XXI) is reacted in step c₂ with an organometallic compound such as a (C₁-C₄)alkylmagnesium halide, in a solvent such as diethyl ether or tetrahydrofuran and at a temperature between −70° C. and ambient temperature.

The resulting compound of formula (XXII) is deprotected in step d₂ according to standard methods (Protective Group in Organic Synthesis, Green et al., 4th Edition, John Wiley & Sons, Inc., New York, 2007).

The compounds of formula (X) in which

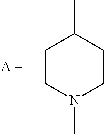

can be prepared according to SCHEME III hereinafter in which Y represents a mesylate and Pg represents a protecting group, preferably a tert-butoxycarbonyl group.

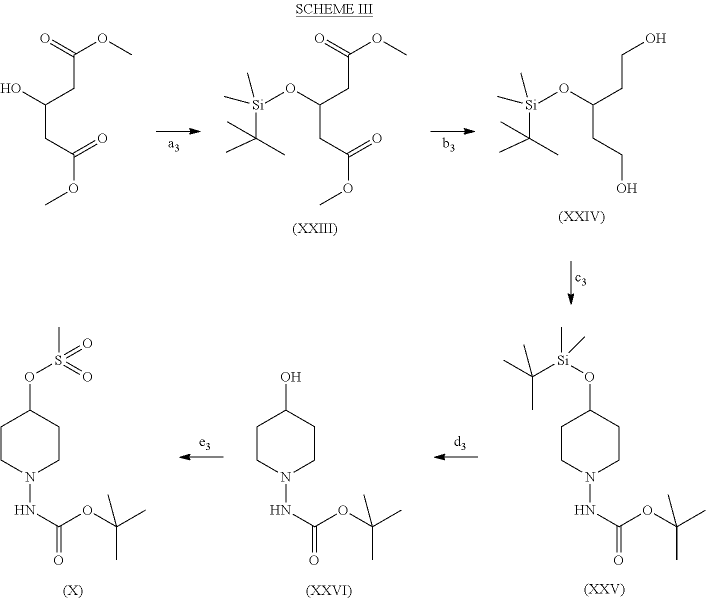

In step a₃ of SCHEME III, the secondary alcohol of dimethyl 3-hydroxyglutarate is protected with a tert-butyldimethylsilyl group.

In step b₃, the resulting compound (XXIII) is reduced in the presence of lithium borohydride in diethyl ether at 0° C. so as to generate the compound (XXIV).

In step c₃, the two alcohol functions are oxidized to aldehyde with a Swern oxidation, then the compound (XXV) is generated by double cyclizing reductive animation with tert-butyl carbazate in the presence of sodium triacetoxyborohydride.

In step d₃, the silylated function is deprotected in order to generate the secondary alcohol of the compound (XXVI).

In step e₃, the alcohol is activated in the presence of methanesulfonyl chloride, triethylamine and 4-dimethylaminopyridine, in dichloromethane, so as to obtain the mesylate (X).

The compounds of formula (IV-A) for which R₃═OZ are prepared by reacting a compound of formula:

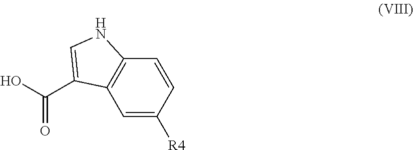

in which R4 is as defined for a compound of formula (I), with a compound of formula (VII):

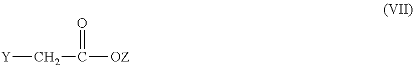

in which Y represents a leaving group such as a halogen atom, a methanesulfonate, a benzenesulfonate, a p-toluenesulfonate or a triflate and Z represents a (C₁-C₄)alkyl.

The reaction is carried out in the presence of two equivalents of a strong base, for instance sodium hydride, in a solvent, in particular an aprotic polar solvent, for instance N,N-dimethylformamide, and at a temperature between −30° C. and the reflux temperature of the solvent.

The compounds of formula (IV-B) are prepared according to SCHEME IV hereinafter in which R4 is as defined for a compound of formula (I):

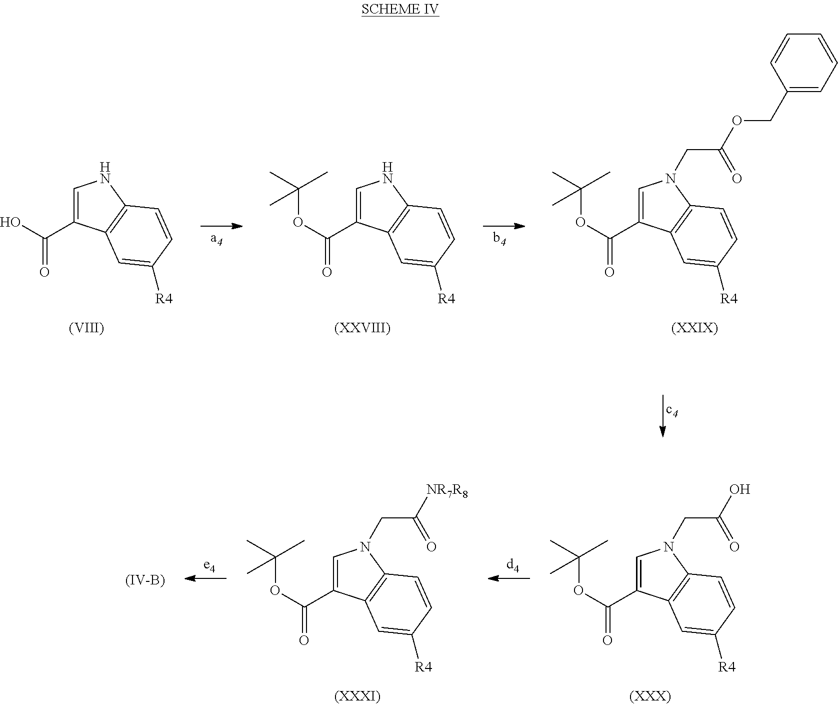

In step a₄ of SCHEME IV, the carboxylic acid of the 3-carboxyindole derivative (VIII) is protected in the presence of N,N-dimethylformamide di-tert-butyl acetal in benzene in order to generate the corresponding tert-butyl ester (XXVIII).

In step b₄, the resulting compound (XXVIII) is alkylated using a compound of formula (VII) below

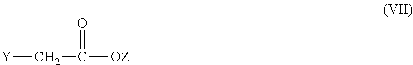

in which Y represents a leaving group such as a halogen atom, a methanesulfonate, a benzenesulfonate, a p-toluenesulfonate or a triflate and Z represents a (C₁-C₄)alkyl or a benzyl group, in the presence of a base such as cesium carbonate and of a solvent such as dimethylformamide.

In step c₄, the ester of the compound (XXIX) is hydrolyzed in the presence of lithium hydroxide at 0° C. so as to obtain the compound (XXX).

In step d₄, the carboxamide bond is generated under activation conditions with the pair 1-(3-dimethylaminopropyl)-3-ethylcarbodiimide/pentafluorophenol in the presence of N-ethylmorpholine and of the desired amine HR₃ so as to give the compound (XXXI).

In step e₄, the tert-butyl ester is deprotected using trifluoroacetic acid in dichloromethane, making it possible to generate the compound (IV-B).

The compounds of formula (VII) are known, commercially available or prepared according to known methods.

The compounds of formula (VIII) may be commercially available or can be prepared according to adaptations of methods described in the literature, such as, for example, those represented in SCHEME V in which R4 is as defined for a compound of formula (I):

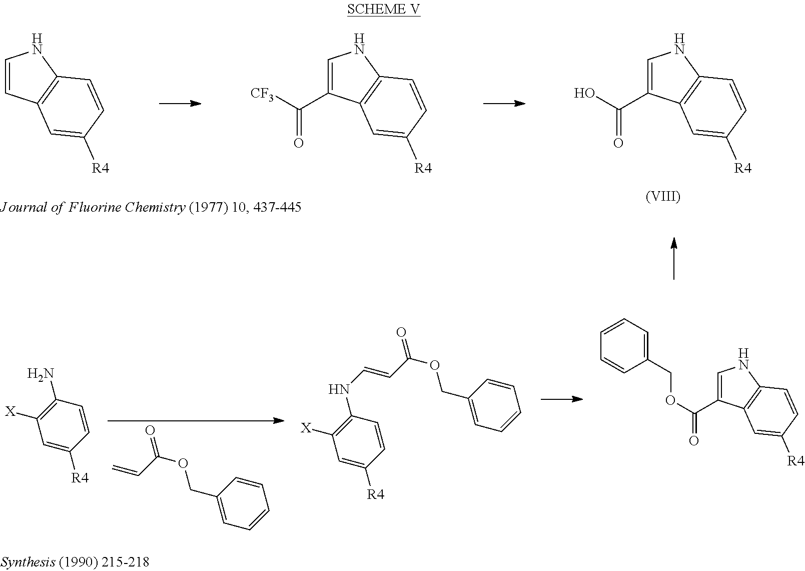

The compounds of formula (IX) in which R₁=(C₁-C₄)alkoxy are known, commercially available or prepared according to known methods (Synlett (2004) 4, 703-707).

According to another of its aspects, a subject of the invention is also the novel compounds of formula (IIA). These compounds are useful as intermediates for synthesizing the compounds of formula (I).

A subject of the invention is also the novel compounds of formula (IIB). These compounds are useful as intermediates for synthesizing the compounds of formula (I).

According to another of its aspects, a subject of the invention is also the use of the compounds of formula (I), as they are or in radiolabeled form as pharmacological tools in humans or in animals, for detecting and labeling the P2Y12 purinergic receptor.

The following examples describe the preparation of certain compounds in accordance with the invention. These examples are not limiting and merely illustrate the present invention.

In the Preparations and in the Examples, the following abbreviations are used:

Me: methyl

Et: ethyl

n-Pr: n-propyl

Ph: phenyl

ether: diethyl ether

iso ether: diisopropyl ether

DMSO: dimethyl sulfoxide

DMF: N,N-dimethylformamide

THF: tetrahydrofuran

DCM: dichloromethane

EtOAc: ethyl acetate

DMAP: 4-dimethylaminopyridine

DIPEA: diisopropylethylamine

HOAT: 1-hydroxy-7-azabenzotriazole

HOBT: 1-hydroxybenzotriazole

TFA: trifluoroacetic acid

BOP-Cl: bis(2-oxo-1,3-oxazolidin-3-yl)phosphinic chloride

EDC: 1-(3-dimethylaminopropyl)-3-ethylcarbodiimide hydrochloride

NaOH: sodium hydroxide

KOH: potassium hydroxide

HCl: hydrochloric acid

i-PrOH: isopropanol

MeCN: acetonitrile

NaBH₄: sodium borohydride

NaHCO₃: sodium hydrogen carbonate

NaH: sodium hydride

Na₂SO₄: sodium sulfate

TBAF: tetra-n-butylammonium fluoride

UV: ultraviolet

1N or 2N hydrochloric ether: 1N or 2N solution of hydrochloric acid in diethyl ether

1N (or 2N) HCl in ether: 1N (or 2N) solution of hydrochloric acid in diethyl ether

4N HCl in dioxane: 4N solution of hydrochloric acid in dioxane

Mp: melting point

AT: ambient temperature

HPLC: high-performance liquid chromatography

Brine: saturated solution of sodium chloride in water.

The proton nuclear magnetic resonance (¹H NMR) spectra are recorded on Bruker (250, 400 and 500 MHz) spectrometers in DMSO-d6. The chemical shifts 6 are expressed in parts per million (ppm). For the interpretation of the spectra, the following abbreviations are used: s: singlet, d: doublet, t: triplet, q: quadruplet: m: unresolved peak, mt: multiplet, bs: broad singlet, dd: split doublet, br: broad peak.

The compounds according to the invention are analyzed by HPLC-UV-MS (liquid chromatography/UV detection/mass detection) coupling.

The apparatus used is composed of a chromatographic system equipped with a diode array detector and a quadrupole mass spectrometer. The molecular peak (MH⁺) and the retention time (tR) in minutes are measured.

Method A: Phenomenex Luna C18(2) column 10×2 mm, 3 μm

Solvent A: water+0.05% TFA

Solvent B: MeCN

1.1 ml/min, 30° C.; Agilent series 1100 MSD

| Gradient (minutes) | A | B |
|---|---|---|
| 0 | 93 | 7 |
| 1.2 | 5 | 95 |
| 1.4 | 5 | 95 |

Method B: Waters XBridge C18 4.6×50 mm, 2.5 μm,

Solvent A: water+0.1% formic acid

Solvent B: MeCN+0.08% formic acid

1.3 ml/min; 20° C.; Waters Ultima Triple Quad MS

| Gradient (minutes) | A | B |
|---|---|---|
| 0 | 97 | 3 |
| 3.5 | 40 | 60 |
| 4 | 2 | 98 |
| 5 | 2 | 98 |
| 5.2 | 97 | 3 |
| 6.5 | 97 | 3 |

Method C: Merck Chromolith C18 2×50 mm; 2.5 μm.

Solvent A: water+0.05% TFA

Solvent B: MeCN+0.05% TFA

2.4 ml/min; 20° C.; Tecan LCT

| Gradient (minutes) | A | B |
|---|---|---|
| 0 | 98 | 2 |
| 0.2 | 98 | 2 |
| 2.4 | 2 | 98 |
| 3.2 | 2 | 98 |
| 4 | 98 | 2 |

Method D: Agilent 1100 series. Symmetry C18 3.5 μm (2.1×50 mm, Waters)

Solvent A: water+0.005% TFA

Solvent B: MeCN+0.005% TFA

0.4 ml/min, 25° C.; MSD SL (Agilent) ESI⁺.

| Gradient (minutes) | A | B |
|---|---|---|
| 0 | 100 | 0 |
| 10 | 0 | 100 |
| 15 | 0 | 100 |

The registration of the mass spectra is carried out in positive electrospray mode (ESI), in order to observe the ions derived from the protonation of compounds analyzed (MH⁺), or from the formation of adducts with other cations such as Na⁺, K⁺, etc.

### Preparations

1. Preparations of the Compounds of Formula (IX)

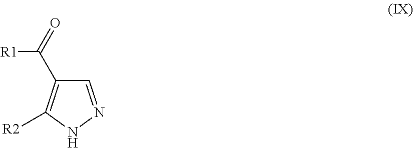

1-(5-methyl-1H-pyrazol-4-yl)butan-1-one

(IX): R1=n-Pr; R2=Me.

### Step 1: 3-methyl-1-trityl-1H-pyrazole-4-carboxylic acid (XX)

8.46 g of potassium carbonate and 15.8 g of trityl chloride are added to a solution of 6.60 g of methyl 5-methyl-1H-pyrazole-4-carboxylate in 50 ml of DMF. After 5 days at AT, EtOAc is added, and the mixture is washed with water and with brine, dried over Na₂SO₄ and evaporated to dryness. The oily residue is suspended in 100 ml of a mixture (50/50; v/v) of ethanol and water. 9.95 g of potassium hydroxide are added and the mixture is heated at reflux for 6 hours. The reaction medium is filtered under hot conditions, and the filtrate is concentrated and acidified with a 1N HCl solution. The precipitate formed is filtered off, washed with a mixture (50/50; v/v) of iso ether and EtOAc and dried under vacuum. 9.7 g of the expected compound are obtained in the form of a white powder.

¹H NMR: DMSO-d₆ (250 MHz): δ (ppm): 2.33 (3H, s); 7.03-7.09 (6H, m); 7.34-7.43 (9H, m); 7.61 (1H, s).

### Step 2: N-methoxy-N,3-dimethyl-1-trityl-1H-pyrazole-4-carboxamide (XXI)

10.3 g of DMAP and 10.3 g of BOP-Cl are added to a solution of 9.70 g of the compound obtained in the previous step, in 100 ml of DCM. 3.85 g of N-methoxymethanamine hydrochloride are then added portionwise and stirred for one hour at AT. Evaporation is carried out and then the residue is taken up with ethyl acetate and the resulting product is washed with water and with brine, dried over Na₂SO₄ and evaporated to dryness. Trituration is carried out with iso ether, filtration is carried out and oven-drying under vacuum is carried out. 10.4 g of the expected compound are obtained in the form of a white powder.

¹H NMR: DMSO-d₆ (250 MHz): δ (ppm): 2.34 (3H, s); 3.13(3H, s); 3.39 (3H, s);

7.05-7.15 (6H, m); 7.33-7.44 (9H, m); 7.71 (1H, s).

### Step 3: 1-(3-methyl-1-trityl-1H-pyrazol-4-yl)butan-1-one (XXII)

32.7 ml of a 2M solution of n-propylmagnesium chloride in ether are added, dropwise, to a solution of 10.4 g of the compound obtained in the previous step, in 130 ml of THF at −30° C. The temperature is brought back to AT and stirring is carried out for 4 hours. The reaction medium is placed at −30° C. and then 50 ml of water are added (dropwise at the beginning). The temperature is brought back to AT, 250 ml of a 1N HCl solution are added and then the mixture is extracted with EtOAc. Washing is carried out with water and brine and the resulting product is dried over Na₂SO₄ and evaporated to dryness. Trituration is carried out from iso ether, filtration is carried out and oven-drying under vacuum is carried out. 8.4 g of the expected compound are obtained in the form of a white powder.

¹H NMR: DMSO-d₆ (400 MHz): δ (ppm): 0.84 (3H, t); 1.52 (2H, sext); 2.34 (3H, s); 2.62 (2H, t); 7.04-7.12 (6H, m); 7.33-7.45 (9H, m); 7.93 (1H, s).

### Step 4: 1-(5-methyl-1H-pyrazol-4-yl)butan-1-one

8.4 g of the compound obtained in the previous step in suspension in 50 ml of 4N HCl in dioxane are stirred for 6 hours. Evaporation to dryness is carried out, trituration is carried out with iso ether, filtration is carried out and oven-drying under vacuum is carried out. 3.1 g of the expected compound are obtained in the form of a colorless gum.

¹H NMR: DMSO-d₆ (250 MHz): δ (ppm): 0.90 (3H, t); 1.58 (2H, sext); 2.39 (3H, s); 2.71 (2H, t); 8.15 (1H, s).

2. Preparations of the Compounds of Formula (V)

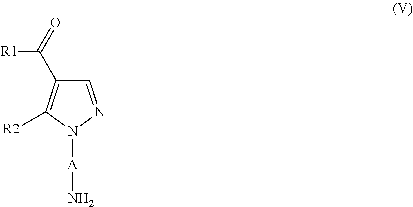

### Preparation 2.1

### 1-[1-(4-Amino-trans-cyclohexyl)-5-methyl-1H-pyrazol-4-yl]-butan-1-one

(V): R1=nPr; R2=Me;

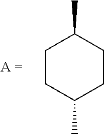

### Step 1: N′-(4-Benzyloxycarbonylaminocyclohexylidene)hydrazinecarboxylic acid tert-butyl ester (XI)

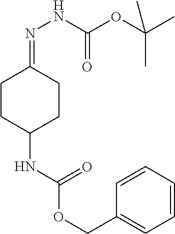 [SMILES: CC(C)(C)OC(=O)NN=C1CCC(NC(=O)OCc2ccccc2)CC1]

11.3 g (44.3 mmol) of benzyl (4-oxocyclohexyl)carbamate and 6.6 g (48.7 mmol) of tert-butyl carbazate are stirred in 140 ml of methanol at AT for 4 h. The reaction medium is evaporated to dryness. The solid residue is triturated with iso ether, and the precipitate is filtered off and then oven-dried under vacuum to give 16.4 g of expected product in the form of a pinkish powder.

Quantitative yield.

¹H NMR: DMSO-d₆ (400 MHz): δ (ppm): 9.54 (1H, br); 7.43-7.24 (6H, m); 5.01 (2H, s); 3.60 (2H, m); 2.74 (1H, m); 2.35-2.11 (2H, m); 2.01-1.76 (2H, m); 1.47-1.25 (11H, m).

### Step 2: N′-(4-Benzyloxycarbonylamino-trans-cyclohexyl)hydrazinecarboxylic acid tert-butyl ester hydrochloride (XII)

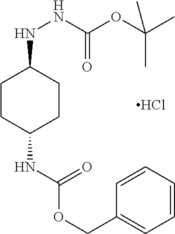 [SMILES: CC(C)(C)OC(=O)NN[C@H]1CC[C@H](NC(=O)OCc2ccccc2)CC1 ; Cl]

16.3 g (45.1 mmol) of N′-(4-benzyloxycarbonylaminocyclohexylidene)hydrazinecarboxylic acid tert-butyl ester prepared in the previous step are suspended in 270 ml of a solution of acetic acid/H₂O (1/1; v/v). 2.78 g (44.2 mmol) of sodium cyanoborohydride are added portionwise and the reaction mixture is stirred at AT overnight. 140 ml of 35% NaOH are slowly added (final pH=6-7). The precipitate formed is filtered off, washed with water (3×) and then oven-dried under vacuum at 60° C. (mixture containing, after NMR analysis, 66% of expected compound in trans configuration and 34% of compound in cis configuration).

The white powder obtained (16.3 g) is dissolved in 370 ml of ethyl acetate. 24.7 ml (49.3 mmol) of a 2N HCl solution in diethyl ether are added dropwise. After stirring overnight, the precipitate is filtered off, washed with acetone and then oven-dried under vacuum at 60° C. to obtain 11.4 g of a white powder (¹H NMR: 100% of the derivative in trans configuration).

Yield=63%

¹H NMR: DMSO-d₆ (400 MHz): δ (ppm): 10.90 (1H, br); 10.18 (1H, br); 7.43-7.19 (6H, m); 5.01 (2H, s); 3.23 (1H, m); 3.06 (1H, m); 1.96 (2H, m); 1.88 (2H, m); 1.49-1.31 (11H, m); 1.18 (2H, m).

### Step 3: (4-Hydrazino-trans-cyclohexyl)carbamic acid benzyl ester hydrochloride (XIII)

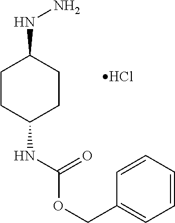 [SMILES: NN[C@H]1CC[C@H](NC(=O)OCc2ccccc2)CC1 ; Cl]

11.3 g (28.3 mmol) of N′-(4-benzyloxycarbonylamino-trans-cyclohexyl)hydrazinecarboxylic acid tert-butyl ester hydrochloride prepared in the previous step are dissolved in 250 ml of dioxane. 93.3 ml of a 4N HCl solution in dioxane (373 mmol) are added dropwise and stirring is maintained for 60 h at AT. The precipitate formed is filtered off and oven-dried under vacuum at 60° C. to obtain 7.61 g of white crystals.

Yield=90%

¹H NMR: DMSO-d₆+TFA (250 MHz): δ (ppm): 7.34 (5H, m); 5.00 (2H, s); 3.24 (1H, m); 2.84 (1H, m); 2.10-1.79 (4H, m); 1.22 (4H, m).

### Step 4: 1-(4-Benzyloxycarbonylamino-trans-cyclohexyl)-5-methyl-1H-pyrazole-4-carboxylic acid ethyl ester (XIV)

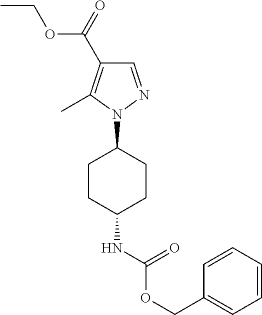 [SMILES: CCOC(=O)c1cnn([C@H]2CC[C@H](NC(=O)OCc3ccccc3)CC2)c1C]

8.83 ml (63.3 mmol) of triethylamine are added to 6.78 g (22.2 mmol) of (4-hydrazino-trans-cyclohexyl)carbamic acid benzyl ester hydrochloride prepared in the previous step in 45 ml of ethanol. 4.6 g of 2-[1-dimethylaminomethylidene]-3-oxobutyric acid ethyl ester are added and the reaction medium is brought to reflux for 4 h. The reaction medium is evaporated to dryness. The residue is taken up with an H₂O/EtOAc mixture. The organic phase is collected and the aqueous phase is extracted with ethyl acetate (3×). The combined organic phases are washed with water (3×) and with brine (1×), dried over Na₂SO₄ and then evaporated to dryness. The residue is triturated with iso ether, filtered and then oven-dried at 60° C. to obtain 6.0 g of expected product in the form of a beige powder.

Yield=74%

¹H NMR: DMSO-d₆ (400 MHz): δ (ppm): 7.77 (1H, s); 7.39-7.27 (6H, m); 5.01 (2H, s); 4.24-4.10 (3H, m); 3.35 (1H, br); 2.52 (3H, s); 1.97-1.80 (6H, m); 1.40 (2H, m); 1.26 (3H, t, J=6.3 Hz).

### Step 5: 1-(4-Benzyloxycarbonylaminocyclohexyl)-5-methyl-1H-pyrazole-4-carboxylic acid (XV)

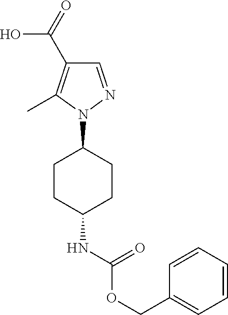 [SMILES: Cc1c(C(=O)O)cnn1[C@H]1CC[C@H](NC(=O)OCc2ccccc2)CC1]

A solution of 3.27 g (58.4 mmol) of potassium hydroxide in water is added to 4.5 g (11.7 mmol) of 1-(4-benzyloxycarbonylamino-trans-cyclohexyl)-5 -methyl-1H-pyrazole-4-carboxylic acid ethyl ester prepared in the previous step in 20 ml of a THF/ethanol mixture (1/1; v/v). The reaction medium is heated at 50° C. overnight. After cooling, water is added and the unsaponifiable compounds are extracted with iso ether. The aqueous phase is acidified at pH 5 using an aqueous solution of hydrochloric acid. The white precipitate formed is filtered off, washed with iso ether and then oven-dried under vacuum at 60° C. to obtain the expected product in the form of a beige powder.

Yield=90%

¹H NMR: DMSO-d₆ (400 MHz): δ (ppm): 12.11 (1H, br); 7.73 (1H, s); 7.39-7.27 (6H, m); 5.01 (2H, s); 4.14 (1H, m); 3.33 (1H, br); 2.50 (3H, s); 1.98-1.81 (6H, m); 1.40 (2H, m).

### Step 6: 3-[1-(4-Benzyloxycarbonylaminocyclohexyl)-5-methyl-1H-pyrazol-4-yl]-3-oxopropionic acid ethyl ester (XVI)

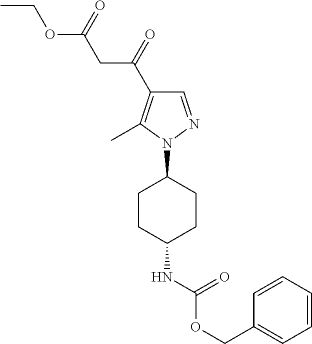 [SMILES: CCOC(=O)CC(=O)c1cnn([C@H]2CC[C@H](NC(=O)OCc3ccccc3)CC2)c1C]

1.97 g (15.9 mmol) of 1,1′-carbonyldiimidazole are added to 3.8 g (10.6 mmol) of 1-(4-benzyloxycarbonylaminocyclohexyl)-5-methyl-1H-pyrazole-4-carboxylic acid in 110 ml of THF. The reaction medium is stirred at AT for 2 h. 4.6 g of magnesium bis(3-ethoxy-3-oxopropanoate) (synthesized according to the method described in Angew. Chem. Int. Ed. Engl., 1979, 18, 72-74) are added and the reaction medium is heated at 50° C. for 3 hours. The reaction mixture is evaporated under vacuum and taken up with EtOAc. The organic phase is washed successively with a saturated solution of Na₂CO₃, with water and with brine, and then dried over Na₂SO₄. The solvent is evaporated under vacuum. The solid residue is triturated with iso ether, filtered and then oven-dried under vacuum at 60° C. to obtain 2.7 g of a pink powder.

Yield=59%

¹H NMR: DMSO-d₆ (400 MHz): δ (ppm): 8.02 (1H, s); 7.39-7.27 (6H, m); 5.01 (2H, s); 4.18 (1H, m); 4.09 (2H, q, J=7 Hz); 3.87 (2H, s); 3.35 (1H, m); 2.53 (3H, s); 1.97-1.79 (6H, m); 1.41 (2H, m); 1.18 (3H, t, J=7 Hz).

### Step 7: 2-[1-(4-Benzyloxycarbonylaminocyclohexyl)-5-methyl-1H-pyrazole-4-carbonyl]butyric acid ethyl ester (VI)

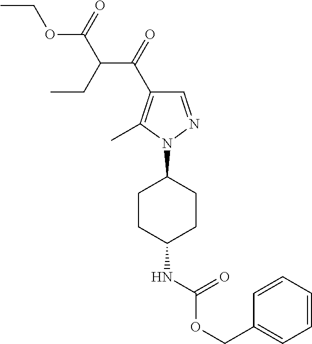 [SMILES: CCOC(=O)C(CC)C(=O)c1cnn([C@H]2CC[C@H](NC(=O)OCc3ccccc3)CC2)c1C]

2.4 g (5.2 mmol) of 3-[1-(4-benzyloxycarbonylaminocyclohexyl)-5-methyl-1H-pyrazol-4-yl]-3-oxopropionic acid ethyl ester prepared in the previous step in 40 ml of THF are placed in a sealed tube. 3.36 g (10.4 mmol) of tetrabutylammonium bromide, 2.85 g (20.7 mmol) of K₂CO₃ and 3.75 ml (38.7 mmol) of iodoethane are added and the reaction medium is heated at 50° C. overnight. The reaction mixture is evaporated to dryness and then taken up with EtOAc. The organic phase is washed with water (1×), with a saturated NaHCO₃ solution (2×) and with brine (1×), and then dried over Na₂SO₄ and evaporated under vacuum. The solid residue is triturated with iso ether, filtered and then oven-dried under vacuum at 60° C. to obtain 3.1 g of a beige powder containing the expected product and one mol of an impurity corresponding to a tetrabutylammonium derivative. The reaction crude is used in the next step (approximate yield of expected product=82%).

¹H NMR: DMSO-d₆ (400 MHz): δ (ppm): 8.09 (1H, s); 7.39-7.27 (6H, m); 5.01 (2H, s); 4.24-4.02 (4H, m); 3.35 (1H, m); 2.53 (3H, s); 1.97-1.72 (8H, m); 1.40 (2H, m); 1.12 (3H, t, J=7 Hz); 0.87 (3H, t, J=7 Hz).

### Step 7: 1-[1-(4-Aminocyclohexyl)-5-methyl-1H-pyrazol-4-yl]butan-1-one

(V): R1=nPr; R2=Me;

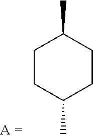

0.6 g of the reaction crude obtained in the previous step (containing ˜0.37 g, i.e. 0.81 mmol, of 2-[1-(4-benzyloxycarbonylaminocyclohexyl)-5-methyl-1H-pyrazole-4-carbonyl]butyric acid ethyl ester is heated at reflux in 6N HCl for 4 h. After cooling, 10 ml of an aqueous 35% sodium hydroxide solution are added (the final pH must be basic). The aqueous phase is extracted with DCM (3×). The combined organic phases are washed successively with water (1×), with a saturated NaHCO₃ solution (1×) and with brine, and then dried over Na₂SO₄ and evaporated to dryness. The solid residue is triturated with iso ether, filtered and then oven-dried under vacuum at 60° C. to obtain 0.6 g of a beige powder containing the expected product and one mol of an impurity corresponding to a tetrabutylammonium derivative (NMR difficult to deconvolve). The reaction crude is used as it is for the next step.

### Preparation 2.2

### 1-[1-(1-Amino-piperidin-4-yl)-5-methyl-1H-pyrazol-4-yl]butan-1-one

(V): R1=n-Pr; R2=Me;

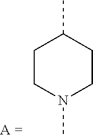

### Step 1: 3-(tert-Butyldimethylsilanyloxy)pentanedioic acid dimethyl ester (XXIII)

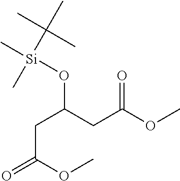

A solution containing 30 g (0.17 mol) of dimethyl 3-hydroxyglutarate in 225 ml of DCM is slowly added to a solution containing 28.2 g (0.19 mol) of tert-butylchlorodimethylsilane and 30.2 g (0.44 mol) of imidazole in 225 ml of DCM. After stirring overnight, water is added, the phases are separated and the aqueous phase is extracted with DCM (2x). The combined organic phases are dried over MgSO₄ and then evaporated to dryness. The residue is purified by silica gel chromatography, eluting with an ethyl acetate/heptane gradient. 40.3 g of the expected compound are obtained in the form of a powder.

Yield=82%

LC-MS tR(method A): 1.10 min; MS (ES) m/z: 291.3 (MH+).

¹H NMR: DMSO-d₆ (500 MHz): δ (ppm): 0.00 (6H, s); 0.70 (9H, s); 3.62 (4H, 2× d); 4.50 (1H, pent).

### Step 2: 3-(tert-Butyldimethylsilanyloxy)pentane-1,5-diol (XXIV)

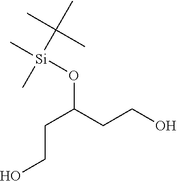

6.0 g (275 mmol) of lithium borohydride are added portionwise to a solution containing 13.5 g (47 mmol) of 3-(tert-butyldimethylsilanyloxy)pentanedioic acid dimethyl ester in 750 ml of diethyl ether at 0° C. After stirring overnight, a saturated aqueous solution of ammonium chloride is added. The aqueous phase is extracted with DCM (2×) and then the organic phases are combined and evaporated to dryness to give 11.9 g of crude product.

Yield˜quantitative.

LC-MS tR(method A): 0.81 min; MS (ES) m/z: 235.3 (MH+).

¹H NMR: DMSO-d₆ (500 MHz): δ (ppm): 0.00 (6H, s); 0.82 (9H, s); 1.52 (4H, m); 3.41 (4H, m); 3.86 (1H, pent); 4.28 (2H, t).

### Step 3: [4-(tert-Butyldimethylsilanyloxy)piperidin-1-yl]carbamic acid tert-butyl ester (XXV)

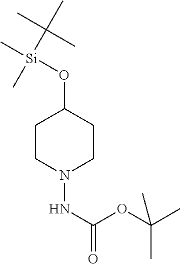 [SMILES: CC(C)(C)OC(=O)NN1CCC(O[Si](C)(C)C(C)(C)C)CC1]

A solution containing 14.4 ml of dimethyl sulfoxide (203 mmol) in 160 ml of DSM is added dropwise, at −78° C., to a solution containing 13.3 ml (155 mmol) of oxalyl chloride in 700 ml of DCM. After stirring for 30 minutes, a solution containing 11.9 g (51 mmol) of 3-(tert-butyldimethylsilanyloxy)pentane-1,5-diol in 160 ml of DCM is added at −78° C. After stirring for a further 30 minutes, 70.7 ml (51 mmol) of triethylamine are added. The reaction medium is stirred at −78° C. for 1 h, then the temperature is brought to 0° C. for 30 minutes of further stirring. 92 ml of toluene are added, then the mixture is filtered. The filtrate is concentrated and then resuspended in pentane and filtered over Celite® to give 13.5 g of crude product after evaporation of the solvent.

12 g of the previously obtained reaction crude are mixed with 7.6 g (58 mmol) of tert-butyl carbazate in 350 ml of DCM. 26.5 g (125 mmol) of sodium triacetoxyborohydride are added at 0° C. and then the reaction medium is stirred overnight. A saturated aqueous solution of NaHCO₃ is added and the phases are separated. The aqueous phase is extracted with DCM (2×) and then the combined organic phases are evaporated to dryness. The residue is purified by silica gel chromatography, eluting with an ethyl acetate/heptane gradient, to obtain 8.6 g of expected product.

Yield=51%.

LC-MS tR(method A): 1.03 min; MS (ES) m/z: 331.3 (MH+).

### Step 4: (4-Hydroxypiperidin-1-yl)carbamic acid tert-butyl ester (XXVI)

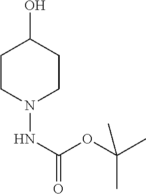

28.6 ml (28.6 mmol) of TBAF (1 M in THF) at 0° C. are added to a solution containing 8.6 g (26 mmol) of [4-(tert-butyldimethylsilanyloxy)piperidin-1-yl]carbamic acid tert-butyl ester in 400 ml of THF. After stirring for 12 h, 28.6 ml of TBAF are added and the reaction medium is stirred for a further 12 h. After concentration to dryness, the residue is redissolved in DCM and extracted with water (3×). The combined aqueous phases are re-extracted with a DCM/i-PrOH mixture (3:1) and the combined organic phases are evaporated to dryness. The residue is purified by silica gel chromatography, eluting with an ethyl acetate/heptane gradient, to obtain 4.4 g of expected product.

Yield=78%.

LC-MS tR(method A): 0.25 min; MS (ES) m/z: 217.3 (MH+).

¹H NMR: DMSO-d₆ (500 MHz): δ (ppm): 1.37 (9H, s); 1.43 (2H, m); 1.69 (2H, m); 2.53 (2H, m); 2.82 (2H, m); 3.47 (1H, m); 4.62 (1H, d); 7.97 (1H, d).

### Step 5: Methanesulfonic acid 1-tert-butoxycarbonylaminopiperidin-4-yl ester

(X: Y═—OSO₂Me;

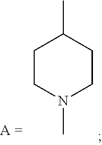

Pg=tert-butoxycarbonyl)

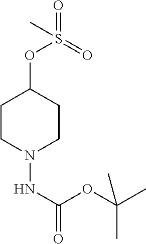 [SMILES: CC(C)(C)OC(=O)NN1CCC(OS(C)(=O)=O)CC1]

0.24 g (2.0 mmol) of DMAP, 2.8 ml (20 mmol) of triethylamine and 1.5 ml (20 mmol) of methanesulfonyl chloride are added to a solution containing 4.3 g (20 mmol) of (4-hydroxypiperidin-1-yl)carbamic acid tert-butyl ester in 100 ml of DCM. After stirring overnight, the reaction medium is diluted with DCM and washed with an aqueous 0.1 M HCl solution. The aqueous phase is extracted with DCM and the organic phases are combined to give 6.0 g of a colorless oil.

Yield=quantitative.

LC-MS tR(method A): 0.61 min; MS (ES) m/z: 239.2 (M-tBu+H+).

¹H NMR: DMSO-d₆ (500 MHz): δ (ppm): 1.40 (9H, s); 1.83 (2H, m); 1.95 (2H, m); 2.78 (2H, m); 2.92 (2H, m); 3.24 (3H, s); 4.76 (1H, m); 8.39 (1H, d).

### Step 6: [4-(4-Butyryl-5-methylpyrazol-1-yl)piperidin-1-yl]carbamic acid tert-butyl ester

(VI: R1=nPr; R2=Me;

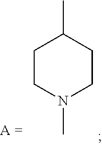

Pg=tert-butoxycarbonyl)

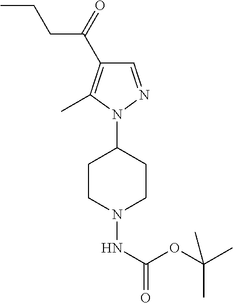 [SMILES: CCCC(=O)c1cnn(C2CCN(NC(=O)OC(C)(C)C)CC2)c1C]

1.20 g (11 mmol) of potassium tert-butoxide are added to a solution containing 1.55 g (10 mmol) of 1-(5-methyl-1H-pyrazol-4-yl)butan-1-one in 15 ml of DMF and the reaction mixture is stirred at 50° C. for 30 minutes. A solution containing 3.00 g (10 mmol) of methanesulfonic acid 1-tert-butoxycarbonylaminopiperidin-4-yl ester in 5 ml of DMF is added. In order to increase the conversion, 0.9 g of potassium tert-butoxide is added in a proportion of 0.3 g every 12 h. The medium is concentrated, diluted with DCM and extracted with water (3×). The crude thus obtained is purified by preparative HPLC (C18 reverse-phase column with elution using a gradient of water/MeCN in the presence of 0.1% of TFA) so as to obtain, after lyophilization, 960 mg of a mixture containing the expected product and its positional isomer. An additional purification by HPLC on chiral stationary phase made it possible to isolate 360 mg of expected product.

Yield=10%.

LC-MS tR(method A): 0.82 min; MS (ES) m/z: 351.2 (MH+).

¹H NMR: DMSO-d₆ (500 MHz): δ (ppm): 0.87 (3H, t); 1.39 (9H, s); 1.57 (2H, m); 1.82 (2H, br d); 2.13 (2H, m); 2.54 (3H, s); 2.73 (2H, t); 2.83 (2H, br t); 3.08 (2H, br d); 4.26 (1H, m); 8.14 (1H, s); 8.61 (1H, br s).

### Step 7: 1-[1-(1-Aminopiperidin-4-yl)-5-methyl-1H-pyrazol-4-yl]butan-1-one hydrochloride

(V: R1=n-Pr; R2=Me;

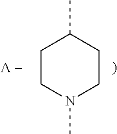

3.3 ml of trifluoroacetic acid are added to a solution containing 536 mg (1.53 mmol) of [4-(4-butyryl-5-methylpyrazol-1-yl)piperidin-1-yl]carbamic acid tert-butyl ester in 12 ml of DCM. After stirring for 3 h, the reaction medium is concentrated and then codistilled twice with toluene. The resulting residue is dissolved in an MeCN/water mixture (1:1) and then lyophilized after addition of 2.0 ml (4 mmol) of 2N HCl. This procedure is repeated once to give 350 mg of expected product in the form of a hydrochloride.

Yield=80%.

LC-MS tR(method A): 0.47 min; MS (ES) m/z: 251.3 (MH+).

¹H NMR: DMSO-d₆ (500 MHz): δ (ppm): 0.87 (3H, t), 1.55 (2H, m), 1.94 (2H, br d), 2.15 (2H, m); 2.55 (3H, s); 2.72 (2H, t); 2.90 (2H, br s); 3.33 (2H, br d); 4.43 (1H, m); 8.10 (1H, s).

3. Preparations of the Compounds of Formula (IV)

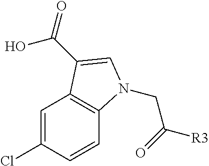

### Preparation 3.1

### 5-Chloro-1-(2-methoxy-2-oxoethyl)-1H-indole-3-carboxylic acid

(IV-A): R3=OMe; R4=Cl

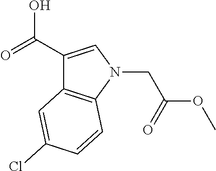 [SMILES: COC(=O)Cn1cc(C(=O)O)c2cc(Cl)ccc21]

110 ml of a solution containing 10 g (51 mmol) of (commercial) 5-chloro-1H-indole-3-carboxylic acid in DMF are added dropwise to a mixture of 4.50 g (112 mmol) of NaH (60% in oil) in 400 ml of DMF at −10° C., and the mixture is left to stir for 1 h. The reaction mixture is cooled to −20° C. and then 4.86 ml (51 mmol) of methyl bromoacetate are added dropwise. The temperature is brought back up to AT over a period of 5 hours and then the reaction medium is stirred at AT for 15 h. The reaction medium is added to 1 l of an EtOAc/1N HCl mixture, the organic phase is collected and the aqueous phase is extracted with EtOAc. The organic phases are combined, washed with water and with brine, and then dried over Na₂SO₄ and evaporated to dryness to obtain 8.9 g of the expected compound in the form of a white powder.

Yield=65%

¹H NMR: DMSO-d₆ (250 MHz): δ (ppm): 3.70 (3H, s); 7.26 (1H, d); 7.57 (1H, d); 7.98 (1H,s); 8.12 (1H, s); 12.3 (1H, br).

### Preparation 3.2

### 5-Chloro-1-[2-(4-methylpiperazin-1-yl)-2-oxoethyl]-1H-indole-3-carboxylic acid (IV-B):

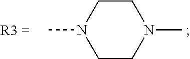

### R4=Cl

### Step 1: 5-Chloro-1H-indole-3-carboxylic acid tert-butyl ester (XXVIII)

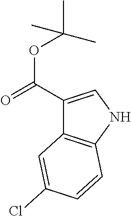 [SMILES: CC(C)(C)OC(=O)c1c[nH]c2ccc(Cl)cc12]

25 ml (104 mmol) of N,N-dimethylformamide di-tert-butyl acetal are added to a solution containing 5.0 g (26 mmol) of 5-chloro-1H-indole-3-carboxylic acid in 200 ml of benzene. The reaction medium is stirred for 12 h and then another portion of the acetal (25 ml) is added. After a further 12 h of stirring, the medium is concentrated, diluted with DCM and then washed with a saturated NaHCO₃ solution (3×). 5.7 g of expected product are obtained after evaporation of the solvents.

Yield=87%.

LC-MS tR(method A): 1.05 min; MS (ES) m/z: 196.1 (M-tBu+H+).

### Step 2: 1-Benzyloxycarbonylmethyl-5-chloro-1H-indole-3-carboxylic acid tert-butyl ester (XXIX)

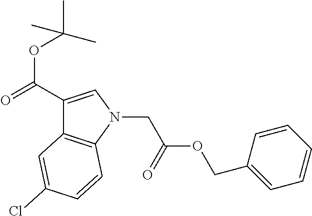 [SMILES: CC(C)(C)OC(=O)c1cn(CC(=O)OCc2ccccc2)c2ccc(Cl)cc12]

11.1 g (34 mmol) of cesium carbonate are added to a solution containing 5.7 g (22 mmol) of 5-chloro-1H-indole-3-carboxylic acid tert-butyl ester and 3.5 ml (22 mmol) of benzyl bromoacetate in 150 ml of DMF. After stirring for 12 h, the reaction medium is diluted with DCM and then washed 3× with an aqueous solution of LiCl (4% w/w). The organic phase is dried over MgSO₄ and the residue obtained after evaporation is purified by silica gel chromatography, eluting with an ethyl acetate/methanol gradient to obtain 5.8 g of expected product.

Yield=66%

LC-MS tR(method A): 1.22 min; MS (ES) m/z: 344.2 (M-tBu+H+).

### Step 3: 1-Carboxymethyl-5-chloro-1H-indole-3-carboxylic acid tert-butyl ester (XXX)

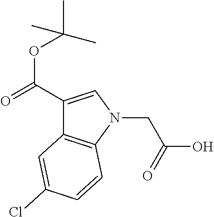 [SMILES: CC(C)(C)OC(=O)c1cn(CC(=O)O)c2ccc(Cl)cc12]

12.2 ml (24 mmol) of a 2M LiOH solution at 0° C. are added to a solution containing 9.8 g (24 mmol) of 5-chloro-1H-indole-3-carboxylic acid tert-butyl ester in a mixture of 140 ml of THF/water (4:1). After stirring for 3 h, the pH of the reaction medium is adjusted to 6 by adding 1N HCl. During the evaporation of the solvents, a solid forms, which is collected by filtration to give 4.3 g of a white powder. Extraction of the filtrate made it possible to obtain a further 4 g of expected product.

Yield=quantitative.

LC-MS tR(method A): 0.96 min; MS (ES) m/z: 254.2 (M-tBu+H+).

### Step 4: 5-Chloro-1-[2-(4-methylpiperazin-1-yl)-2-oxoethyl]-1H-indole-3-carboxylic acid tert-butyl ester

(XXXI:

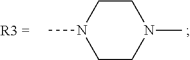

### R4=Cl)

2.93 g (20 mmol) of EDC and 2.81 g (15 mmol) of pentafluorophenol are added to a solution containing 3.15 g (10 mmol) of 1-carboxymethyl-5-chloro-1H-indole-3-carboxylic acid tert-butyl ester in 70 ml of DMF. After stirring for 20 minutes, 3.8 ml (30 mmol) of N-ethylmorphine and 1.1 ml (10 mmol) of 1-methylpiperazine are added and the reaction medium is stirred for 12 h at AT. After evaporation of the solvent, the residue is taken up with DCM and then washed 2× with an aqueous solution of LiCl (4% w/w). The organic phase is dried over MgSO₄ and evaporated to dryness and then the crude product obtained is purified by silica gel chromatography, eluting with an ethyl acetate/methanol gradient to obtain 2.26 g of expected product.

Yield=58%.

LC-MS tR(method A): 0.73 min; MS (ES) m/z: 336.2 (M-tBu+H+).

¹H NMR: DMSO-d₆ (500 MHz): δ (ppm): 1.58 (9H, s); 2.24 (3H, s); 2.31 (2H, m); 2.44 (2H, m); 3.47 (2H, m); 3.58 (2H, m); 5.34 (2H, s); 7.28 (1H, dd); 7.54 (1H, d); 7.99 (1H, d); 8.05 (1H, s).

### Step 5: 5-Chloro-1-[2-(4-methylpiperazin-1l-yl)-2-oxoethyl]-1H-indole-3-carboxylic acid

(IV-B:

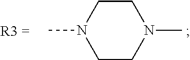

### R4=Cl)

4.3 ml of trifluoroacetic acid are added to a solution containing 2.26 g (5.8 mmol) of 5 -chloro-1-[2-(4-methylpiperazin-1-yl)-2-oxoethyl]-1H-indole-3-carboxylic acid tert-butyl ester in 30 ml of DCM. After stirring for 3 h, the reaction medium is concentrated and then codistilled with toluene (2×). The resulting residue is dissolved in an MeCN/water mixture and lyophilized after addition of 7.3 ml of 2N HCl. This procedure is repeated once to obtain 2.33 g of the hydrochloride of the expected product.

Yield=quantitative.

LC-MS tR(method A): 0.51 min; MS (ES) m/z: 336.2 (MH+).

¹H NMR: DMSO-d₆ (500 MHz): δ (ppm): 2.79 (3H, s); 2.96 (1H, m); 3.13 (2H, m); 3.43 (1H, d); 3.52 (1H, d); 3.62 (1H, t); 4.15 (1H, d); 4.37 (1H, d); 5.34 (1H, d); 5.50 (1H, d); 7.31 (1H, dd); 7.61 (1H, d); 8.04 (1H, d); 8.06 (1H, s); 11.25 (1H, br s).

### Preparation 3.3

### 5-Chloro-1-{2-[4-(2-methoxyethyl)piperazin-1-yl]-2-oxoethyl}-1H-indole-3-carboxylic acid

(IV-B):

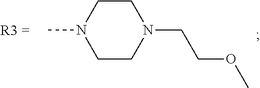

### R4=Cl

### Step 1: 5-Chloro-1-{2-[4-(2-methoxyethyl)piperazin-1-yl]-2-oxoethyl}-1H-indole-3-carboxylic acid tert-butyl ester

(XXXI:

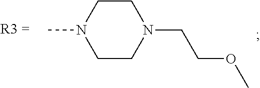

### R4=Cl)

3.99 g (28 mmol) of EDC and 3.83 g (21 mmol) of pentafluorophenol are added to a solution containing 4.3 g (14 mmol) of 1-carboxymethyl-5-chloro-1H-indole-3-carboxylic acid tert-butyl ester in 95 ml of DMF. After stirring for 20 minutes, 5.5 ml (42 mmol) of N-ethylmorpholine and 2.1 ml (14 mmol) of 1-(2-methoxyethyl)piperazine are added and the reaction medium is stirred for 12 h at AT. After evaporation of the solvent, the residue is taken up with DCM and then washed 2× with an aqueous solution of LiCl (4% w/w). The organic phase is dried over MgSO₄ and evaporated to dryness and then the crude product obtained is purified by silica gel chromatography, eluting with an ethyl acetate/methanol gradient to obtain 5.4 g of expected product.

Yield=87%.

LC-MS tR(method A): 0.75 min; MS (ES) m/z: 436.4 (MH+).

### Step 2: 5-Chloro-1-{2-[4-(2-methoxyethyl)piperazin-1-yl]-2-oxoethyl}-1H-indole-3-carboxylic acid

(IV-B:

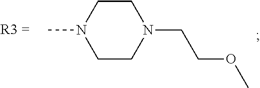

### R4=Cl)

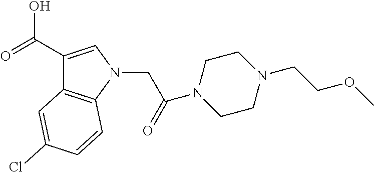 [SMILES: COCCN1CCN(C(=O)Cn2cc(C(=O)O)c3cc(Cl)ccc32)CC1]

3.4 ml of trifluoroacetic acid are added to a solution containing 2 g (4 6 mmol) of 5-chloro-1-{2-[4-(2-methoxyethyl)piperazin-1-yl]-2-oxoethyl}-1H-indole-3-carboxylic acid tert-butyl ester in 25 ml of DCM. After stirring for 3 h, the reaction medium is concentrated and then codistilled with toluene (2×). The resulting residue is dissolved in an MeCN/water mixture and lyophilized after addition of 6.5 ml of 1N HCl. This procedure is repeated once to obtain 1.92 g of the hydrochloride of the expected product.

Yield=quantitative.

LC-MS tR(method A): 0.55 min; MS (ES) m/z: 380.3 (MH+).

### Preparation 3.4

### 5-Chloro-1-{2-[4-(2-methoxyethyl)piperazin-1-yl]-2-oxoethyl}-1H-indole-3-carboxylic acid

(IV-B):

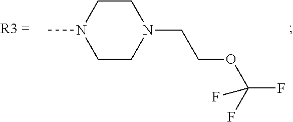

### R4=Cl

### Step 1: 1-Benzyl-4-(2-trifluoromethoxyethyl)piperazine

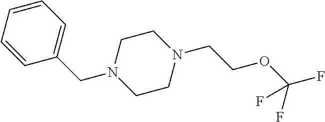 [SMILES: FC(F)(F)OCCN1CCN(Cc2ccccc2)CC1]

A suspension of 0.77 g (4.7 mmol) of 2-(trifluoromethoxy)ethylamine hydrochloride and 1.8 g (21 mmol) of NaHCO₃ in 100 ml of ethanol is stirred at 80° C. for 6 h. The reaction medium is filtered and concentrated. The residue is taken up with DCM and washed with water (1×), and then the aqueous phase is re-extracted with DCM. The combined organic phases are evaporated and then the crude product is purified by silica gel chromatography, eluting with an ethyl acetate/heptane gradient to obtain 419 mg of expected product in the form of a colorless oil.

Yield=31%.

LC-MS tR(method A): 0.46 min; MS (ES) m/z: 289.3 (MH+).

¹H NMR: DMSO-d₆ (500 MHz): δ (ppm): 2.33-2.52 (8H, m); 2.65 (2H, t); 3.52 (2H, s); 4.21 (2H, t); 7.31-7.41 (5H, m).

### Step 2: 1-(2-Trifluoromethoxyethyl)piperazine

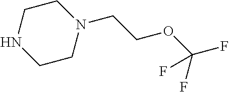 [SMILES: FC(F)(F)OCCN1CCNCC1]

A solution of 2.20 g (7.6 mmol) of 1-benzyl-4-(2-trifluoromethoxyethyl)piperazine in 50 ml of THF is transferred into a Büchi autoclave and then hydrogenated for 9 h at 3 bar in the presence of 200 mg (0.29 mmol) of Pd(OH)₂ at 20% on carbon. Since the conversion is not complete, the suspension is filtered, fresh catalyst is added and the reaction medium is hydrogenated for a further 3 h. After filtration over Celite®, washing with ethanol and evaporation to dryness, 973 mg of debenzylated product are obtained in the form of a colorless oil.

Yield=65%.

LC-MS tR(method A): 0.09 min; MS (ES) m/z: 199.2 (MH+).

¹H NMR: CDCl3 (400 MHz): δ (ppm): 2.53-2.65 (4H, m); 2.75 (2H, t); 3.02 (4H, m); 3.59 (1H, m); 4.15 (2H, t).

### Step 3: 5-Chloro-1-{2-oxo-2-[4-(2-trifluoromethoxyethyl)piperazin-1-yl]ethyl}-1H-indole-3-carboxylic acid tert-butyl ester

(XXXI):

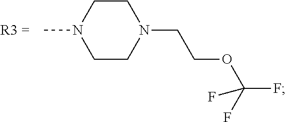

### R4=Cl

557 mg (3.8 mmol) of EDC and 534 mg (2 9 mmol) of pentafluorophenol are added to a solution containing 600 mg (1.9 mmol) of 1-carboxymethyl-5-chloro-1H-indole-3-carboxylic acid tert-butyl ester in 30 ml of DMF. After stirring for 20 minutes, 0.7 ml (5.7 mmol) of N-ethylmorpholine and 416 mg (2.0 mmol) of 1-(2-trifluoromethoxyethyl)piperazine are added and the reaction medium is stirred for 12 h at AT. After evaporation of the solvent, the residue is taken up with DCM and then washed 2× with an aqueous solution of LiCl (4% w/w). The organic phase is dried over MgSO₄ and evaporated to dryness and then the crude product obtained is purified by silica gel chromatography, eluting with an ethyl acetate/heptane gradient to obtain 634 mg of expected product.

Yield=68%.

LC-MS tR(method A): 0.75 min; MS (ES) m/z: 436.4 (MH+).

### Step 4: 5-Chloro-1-{2-oxo-2-[4-(2-trifluoromethoxyethyl)piperazin-1-yl]ethyl}-1H-indole-3-carboxylic acid

(IV-B):

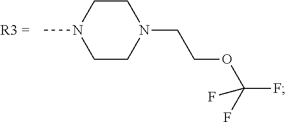

### R4=Cl

0.9 ml of trifluoroacetic acid are added to a solution containing 634 mg (1.4 mmol) of 5-chloro-1-{2-[4-(2-methoxyethyl)piperazin-1-yl]-2-oxoethyl}-1H-indole-3-carboxylic acid tert-butyl ester in 10 ml of DCM. After stirring for 3 h, the reaction medium is concentrated and then codistilled with toluene (2×). The resulting residue is dissolved in an MeCN/water mixture and lyophilized after addition of 1.3 ml of 2N HCl. This procedure is repeated once to obtain 567 mg of the hydrochloride of the expected product.

Yield=94%.

LC-MS tR(method A): 0.55 min; MS (ES) m/z: 380.3 (MH+).

### 4 Preparation of the compound {3-[4-(4-butyryl-5-methylpyrazol-1-yl)-trans-cyclohexylcarbamoyl]-5-chloroindo]-1-yl}acetic acid

(IIB: R1=nPr; R2=Me;

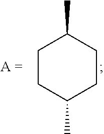

### R3=OH; R4=Cl)

### Step 1: {3-[4-(4-Butyryl-5-methylpyrazol-1-yl)-trans-cyclohexylcarbamoyl]-5-chloroindol-1-yl}acetic acid methyl ester

(IIA: R1=nPr; R2=Me;

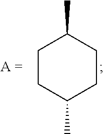

### R3=OZ; R4=Cl; Z=Me)

439 mg (2.2 mmol) of EDC are added to a solution containing 600 mg (2.0 mmol) of 5-chloro-l-methoxycarbonylmethyl-1H-indole-3-carboxylic acid (preparation 2.1) and 528 mg (4.3 mmol) of DMAP in 12 ml of DCM. After stirring for a few minutes, the medium becomes homogeneous and 610 mg (2.45 mmol) of 1-[1-(4-amino-trans-cyclohexyl)-5-methyl-1H-pyrazol-4-yl]butan-1-one (preparation 1.1) are added. After stirring at AT for 48 h, the reaction medium is washed with water (1×), with 1N HCl (1×), with NaHCO₃ (1×) and then with brine. The organic phase is dried over Na₂SO₄ and then evaporated to dryness to obtain 1.1 g of brown oil. The oily residue is purified by silica gel chromatography, eluting with a DCM/methanol gradient. The yellow oil obtained (550 mg) is taken up with acetone and then poured dropwise onto iso ether. The white precipitate formed is filtered off, washed with iso ether and dried under vacuum to obtain 300 mg of a white powder.

Yield=30%.

¹H NMR: DMSO-d₆ (400 MHz): δ (ppm): 8.15 (1H, d, J=2.2 Hz); 8.08 (1H, s); 8.03 (1H, s); 7.93 (1H, d, J=7.7 Hz); 7.52 (1H, d, J=8.9 Hz); 7.21 (1H, dd, J=8.9, 2.2 Hz); 5.25 (2H, s); 4.22 (1H, m); 3.85 (1H, m); 3.70 (3H, s); 2.73 (2H, t, J=7.2 Hz); 2.56 (3H, s); 2.05-1.85 (6H, m); 1.59 (2H, sext, J=7.2 Hz); 1.55 (2H, m); 0.91 (3H, t, J=7.2 Hz).

### Step 2: {3-[4-(4-Butyryl-5-methylpyrazol-1-yl)-trans-cyclohexylcarbamoyl]-5-chloroindol-1-yl}acetic acid

(IIB: R1=nPr; R2=Me;

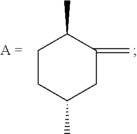

### R3=OH; R4=Cl)

26 mg (0.66 mmol) of NaOH are added to a solution containing 300 mg (0.60 mmol) of {3-[4-(4-butyryl-5-methylpyrazol-1-yl)-trans-cyclohexylcarbamoyl]-5-chloroindol-1-yl}acetic acid methyl ester (prepared in the previous step) in 3 ml of methanol. The reaction medium is stirred for 2 h and then evaporated to dryness. The residue is taken up with water and then 0.7 ml of 1N HCl is added. The precipitate formed is filtered off, washed with water and then oven-dried under vacuum at 60° C. to obtain 260 mg of a white powder.

Yield=89%.

¹H NMR: DMSO-d₆ (400 MHz): δ (ppm): 8.15 (1H, d, J=2.2 Hz); 8.09 (1H, s); 8.03 (1H, s); 7.90 (1H, d, J=7.8 Hz); 7.49 (1H, d, J=8.8 Hz); 7.19 (1H, dd, J=8.8, 2.2 Hz); 5.03 (2H, s); 4.22 (1H, m); 3.85 (1H, m); 2.72 (2H, t, J=7.2 Hz); 2.56 (3H, s); 2.05-1.85 (6H, m); 1.59 (2H, sextuplet, J=7.2 Hz); 1.55 (2H, m); 0.91 (3H, t, J=7.2 Hz).

### EXAMPLES

### Example 1

### Compound No. 1

### 5-Chloro-1-[2-(4-methylpiperazin-1-yl)-2-oxoethyl]-1H-indole-3-carboxylic acid [4-(4-butyryl-5-methyl-pyrazol-1-yl)-cyclohexyl]amide hydrochloride

260 mg (0.54 mmol) of {3-[4-(4-butyryl-5-methylpyrazol-1-yl)-trans-cyclohexylcarbamoyl]-5-chloroindol-1-yl}acetic acid (preparation 3), 136 mg (1.3 mmol) of N-methylpiperazine and 211 mg (0.80 mmol) of BOP-Cl in 5 ml of DCM are stirred overnight. The reaction medium is evaporated to dryness and then the solid residue is triturated with a saturated solution of NaHCO₃. The white precipitate formed is filtered off, washed with water, oven-dried under vacuum at 60° C., and then purified by silica gel chromatography, eluting with a DCM/methanol gradient to obtain 290 mg of a white powder in base form. The product is taken up in acetone and then 0.7 ml of 1N HCl in diethyl ether is added. The medium is filtered and the precipitate is oven-dried under vacuum at 60° C. to obtain 260 mg of a white powder.

Yield=80%.

LC-MS tR(method D): 6.1 min; MS (ES) m/z: 567 (MH+).

Mp=214° C.

¹H NMR: DMSO-d₆ (400 MHz): δ (ppm): 10.80 (1H, br); 8.15 (1H, d, J=2.0 Hz); 8.03 (1H, s); 8.02 (1H, s); 7.92 (1H, d, J=7.7 Hz); 7.49 (d, 1H, J=8.9 Hz); 7.20 (1H, dd, J=8.9, 2.0 Hz); 5.41 (1H, system AB: J=17 Hz); 5.27 (1H, system AB: J=17 Hz); 4.37 (1H, m); 4.20 (2H, m); 3.85 (1H, m); 3.56 (2H, m); 3.45 (1H, m); 3.12 (2H, m); 2.97 (1H, m); 2.83 (3H, s); 2.73 (2H, t, J=7.2 Hz); 2.56 (3H, s); 2.05-1.85 (6H, m); 1.59 (2H, sext, J=7.2 Hz); 1.55 (2H, m); 0.90 (3H, t, J=7.2 Hz).

### Example 2

### Compound No. 2

### 5-Chloro-1-{2-[4-(2-methoxyethyl)piperazin-1-yl]-2-oxoethyl}-1H-indole-3-carboxylic acid [4-(4-butyryl-5-methylpyrazol-1-yl)cyclohexyl]-amide hydrochloride

250 mg (0.52 mmol) of {3-[4-(4-butyryl-5-methylpyrazol-1-yl)-trans-cyclohexylcarbamoyl]-5-chloroindol-1-yl}acetic acid (preparation 3), 112 mg (0.77 mmol) of 1-(2-methoxyethyl)piperazine, 191 mg (1 5 mmol) of DMAP and 203 mg (0.77 mmol) of BOP-Cl in 3.4 ml of DCM are stirred overnight. The reaction medium is evaporated to dryness and then the solid residue is triturated with a saturated solution of NaHCO₃. The precipitate is purified by silica gel chromatography, eluting with a DCM/methanol gradient to obtain 240 mg of a white powder in base form. The product is taken up in acetone and then 0.5 ml of 1N HCl in diethyl ether is added. The medium is filtered and the precipitate is oven-dried under vacuum at 60° C. to obtain 200 mg of a white powder.

Yield=59%.

LC-MS tR(method D): 6.2 min; MS (ES) m/z: 611 (MH+).

Mp=263° C.

¹H NMR: DMSO-d₆ (400 MHz): δ (ppm): 10.56 (1H, br); 8.15 (1H, d, J=2.0 Hz); 8.03 (1H, s); 8.02 (1H, s); 7.91 (1H, d, J=7.7 Hz); 7.49 (d, 1H, J=8.9 Hz); 7.20 (1H, dd, J=8.9, 2.0 Hz); 5.41 (1H, system AB: J=17 Hz); 5.27 (1H, system AB: J=17 Hz); 4.35 (1H, m); 4.20 (2H, m); 3.86 (1H, m); 3.74 (2H, m); 3.70-3.47 (3H, m); 3.38 (2H, m); 3.34 (3H, s); 3.18 (2H, m); 3.02 (1H, m); 2.73 (2H, t, J=7.2 Hz); 2.56 (3H, s); 2.05-1.85 (6H, m); 1.65-1.49 (4H, m); 0.90 (3H, t, J=7.2 Hz).

### Example 3

### Compound No. 3 5-Chloro-1-{2-oxo-2-[4-(3,3,3-trifluoropropyl)piperazin-1-yl]ethyl}-1H-indole-3-carboxylic acid [4-(4-butyryl-5-methylpyrazol-1-yl)cyclohexyl]amide hydrochloride

150 mg (0.31 mmol) of {3-[4-(4-butyryl-5-methylpyrazol-1-yl)-trans-cyclohexylcarbamoyl]-5-chloroindol-1-yl}acetic acid (preparation 3), 118 mg (0.46 mmol) of 1-(3,3,3-trifluoropropyl)piperazine dihydrochloride, 229 mg (1.9 mmol) of DMAP and 122 mg (0.46 mmol) of BOP-Cl in 2 ml of DCM are stirred overnight. The reaction medium is evaporated to dryness and then the solid residue is triturated with a saturated solution of NaHCO₃. The precipitate is purified by silica gel chromatography, eluting with a DCM/methanol gradient to obtain 140 mg of a white powder in base form. The product is taken up in DCM and then 0.28 ml of 1N HCl in diethyl ether is added. The medium is filtered and the precipitate is oven-dried under vacuum at 60° C. to obtain 115 mg of a white powder.

Yield=54%.

LC-MS tR(method D): 6.98 min; MS (ES) m/z: 650 (MH+).

Mp=249° C.

1H NMR: DMSO-d₆ (400 MHz): δ (ppm): 11.41 (1H, br); 8.15 (1H, d, J=2.0 Hz); 8.03 (1H, s); 8.02 (1H, s); 7.90 (1H, d, J=7.7 Hz); 7.48 (d, 1H, J=8.9 Hz); 7.20 (1H, dd, J=8.9, 2.0 Hz); 5.39 (1H, br); 5.30 (1H, br); 4.38 (1H, m); 4.21 (2H, m); 3.86 (1H, m); 3.65 (3H, m); 3.18 (2H, m); 2.98 (3H, m); 2.73 (2H, t, J=7.2 Hz); 2.56 (3H, s); 2.05-1.85 (6H, m); 1.65-1.49 (4H, m); 0.90 (3H, t, J=7.2 Hz).

### Example 4

### Compound No. 4

### 5 -Chloro-1-[2-(4-methylpiperazin-1-yl)-2-oxoethyl]-1H-indole-3 -carboxylic acid [4-(4-butyryl-5 -methylpyrazol-1-yl)piperidin-1-yl]amide hydrochloride

152 mg (1.1 mmol) of HOBt, 0.6 ml (3.5 mmol) of DIPEA and 191 mg (1 mmol) of EDC are added to a solution containing 337 mg (1.0 mmol) of 5-chloro-1-{2-[4-methylpiperazin-1-yl]-2-oxoethyl}-1H-indole-3-carboxylic acid (preparation 2.2) in 45 ml of DMF. After stirring for 10 minutes, 330 mg (1.1 mmol) of 1-[1-(1-aminopiperidin-4-yl)-5-methyl-1H-pyrazol-4-yl]butan-1-one hydrochloride (preparation 1.2) are added and the reaction medium is stirred for 24 h. 95 mg of EDC, 75 mg of HOBt and 0.3 ml of DIPEA are added and, after a further 24 h, the conversion is complete. The reaction medium is evaporated to dryness, and the residue is purified by preparative HPLC (C18 reverse-phase column with elution using an H₂O/MeCN gradient in the presence of 0.1% of TFA). The resulting product is dissolved in an MeCN/water mixture and lyophilized after addition of 0.43 ml of 1N HCl. This procedure is repeated once to obtain 186 mg of the hydrochloride of the expected product.

Yield=33%.

LC-MS tR(method C): 1.26 min; MS (ES) m/z: 568.2 (MH+).

¹H NMR: DMSO-d₆ (500 MHz): δ (ppm): 0.88 (3H, t); 1.57 (2H, dt); 2.05 (2H, m); 2.42 (2H, m); 2.59 (3H, s); 2.75 (2H, t); 2.83 (3H, d); 3.00 (1H, m); 3.19 (2H, m); 3.45-3.71 (6H, m); 4.22 (2H, d); 4.41 (2H, d); 4.57 (1H, br s); 5.43 (1H, d); 5.57 (1H, d); 7.36 (1H, d); 7.66 (1H, d); 8.18 (1H, s); 8.21 (1H, s); 8.32 (1H, s); 11.40 (1H, br s).

### Example 5

### Compound No. 5

### 5-Chloro-1-{2-[4-(2-methoxyethyl)piperazin-1-yl]-2-oxoethyl}-1H-indole-3-carboxylic acid [4-(4-butyryl-5-methylpyrazol-1-yl-piperidin-1-yl]amide hydrochloride

200 mg (1.5 mmol) of HOBt, 1.3 ml (7.5 mmol) of DIPEA and 250 mg (1.3 mmol) of EDC are added to a solution containing 436 mg (1.3 mmol) of 5-chloro-1-{2-[4-(2-methoxyethyl)piperazin-1-yl]-2-oxoethyl}-1H-indole-3-carboxylic acid (preparation 2.3) in 60 ml of DMF. After stirring for 10 minutes, 300 mg (1.2 mmol) of 1-[1-(1-aminopiperidin-4-yl)-5-methyl-1H-pyrazol-4-yl]butan-1-one hydrochloride (preparation 1.2) are added and the reaction medium is stirred for 24 h. 83 mg of EDC, 67 mg of HOBt and 0.4 ml of DIPEA are added and, after a further 24 h, the conversion is complete. The reaction medium is evaporated to dryness, and the residue is dissolved in 250 ml of a DCM/iPrOH mixture (3:1) and then washed with an aqueous solution of LiCl (4% w/w). The organic phase is dried over MgSO₄, and the residue is purified by preparative HPLC (C18 reverse-phase column with elution using an H₂O/MeCN gradient in the presence of 0.1% of TFA). The resulting product is dissolved in an MeCN/water mixture and lyophilized after addition of 1 ml of 1N HCl. This procedure is repeated once to obtain 250 mg of the hydrochloride of the expected product.

Yield=30%.

LC-MS t_{R}(method B): 3.24 min; MS (ES) m/z: 612.3 (MH+).

¹H NMR: DMSO-d₆ (500 MHz): δ (ppm): 0.88 (3H, t); 1.57 (2H, dt); 2.06 (2H, m); 2.43 (2H, m); 2.59 (3H, s); 2.74 (2H, t); 3.06 (1H, m); 3.24 (2H, m); 3.35 (3H, s); 3.39 (2H, d); 3.50-3.81 (8H, m); 4.21 (2H, d); 4.39 (2H, d); 4.59 (1H, br s); 5.43 (1H, d); 5.57 (1H, d); 7.36 (1H, d); 7.67 (1H, d); 8.18 (1H, s); 8.21 (1H, s); 8.35 (1H, s); 11.21 (1H, br s).

### Example 6

### Compound No. 6

### 5-Chloro-1-{2-oxo-2-[4-(2-trifluoromethoxyethyl)piperazin-1-yl]ethyl}-1H-indole-3-carboxylic acid [4-(4-butyryl-5-methylpyrazol-1-yl)piperidin-1-yl]amide hydrochloride

200 mg (1.5 mmol) of HOBt, 1.3 ml (7.5 mmol) of DIPEA and 250 mg (1.3 mmol) of EDC are added to a solution containing 492 mg (1.3 mmol) of 5-chloro-1-{2-[4-(2-methoxyethyl)piperazin-1-yl]-2-oxoethyl}-1H-indole-3-carboxylic acid (preparation 2.4) in 60 ml of DMF. After stirring for 10 minutes, 300 mg (1.2 mmol) of 1-[1-(1-aminopiperidin-4-yl)-5-methyl-1H-pyrazol-4-yl]butan-1-one hydrochloride (preparation 1.2) are added and the reaction medium is stirred for 24 h. 83 mg of EDC, 67 mg of HOBt and 0.4 ml of DIPEA are added and, after a further 24 h, the conversion is complete. The reaction medium is evaporated to dryness, and the residue is dissolved in 250 ml of a DCM/iPrOH mixture (3:1) and then washed with an aqueous solution of LiCl (4% w/w). The organic phase is dried over MgSO₄, and the residue is purified by preparative HPLC (C18 reverse-phase column with elution using an MeCN/water gradient in the presence of 0.1% of TFA). The resulting product is dissolved in an MeCN/water mixture and lyophilized after addition of 1 ml of 1N HCl. This procedure is repeated once to obtain 250 mg of the hydrochloride of the expected product.

Yield=29%.

LC-MS tR(method B): 3.62 min; MS (ES) m/z: 666.3 (MH+).

¹H NMR: DMSO-d₆ (500 MHz): δ (ppm): 0.88 (3H, t); 1.57 (2H, dt); 1.99 (2H, m); 2.34 (2H, m); 2.58 (3H, s); 2.74 (2H, t); 3.04-3.67 (12H, m); 4.25 (1H, d); 4.47 (2H, d); 4.67 (2H, br s); 5.42 (1H, br s); 5.53 (1H, br s); 7.32 (1H, d); 7.62 (1H, d); 8.16 (1H, s); 8.19 (2H, s); 11.52 (1H, br s).

The compounds according to the invention were the subject of pharmacological tests.

Inhibition of Platelet Aggregation In Vitro (Rat Blood)

Blood is taken from male rats of the Sprague-Dawley strain, weighing 250-300 g. The sample is taken on 3.8% sodium citrate (1 volume for 9 blood volumes) by abdominal aortic puncture after the animal has been anesthetized with sodium pentobarbital.

The platelet-rich plasma (PRP) is obtained by centrifugation of the blood at 300 g for 5 minutes, and the platelet aggregation measurements are carried out as described above.

The results are calculated using the area under the curve of absorbance measured for 6 minutes, and expressed by the percentage inhibition.

The compounds according to the invention have IC₅₀ (platelet aggregation inhibition) values of between 0.02 and 1.5 μM.

The results obtained for each compound are indicated in table I.

TABLE 1

| | | Inhibition of platelet |
|---|---|---|
| Compound | | aggregation in vitro (rat blood) |
| No. | Structural formula | IC₅₀ in μM |
| 1 | 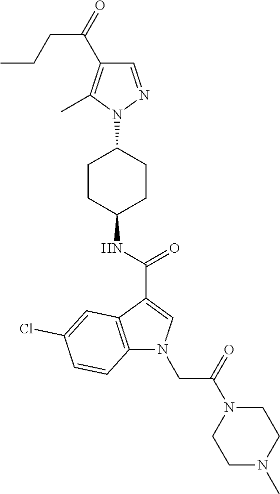 [SMILES: CCCC(=O)c1cnn([C@H]2CC[C@H](NC(=O)c3cn(CC(=O)N4CCN(C)CC4)c4ccc(Cl)cc34)CC2)c1C] | 0.091 |
| 2 | 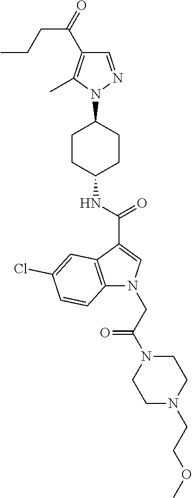 [SMILES: CCCC(=O)c1cnn([C@H]2CC[C@H](NC(=O)c3cn(CC(=O)N4CCN(CCOC)CC4)c4ccc(Cl)cc34)CC2)c1C] | 0.080 |
| 3 | 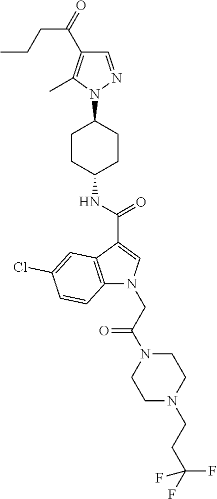 [SMILES: CCCC(=O)c1cnn([C@H]2CC[C@H](NC(=O)c3cn(CC(=O)N4CCN(CCC(F)(F)F)CC4)c4ccc(Cl)cc34)CC2)c1C] | 0.294 |
| 4 | 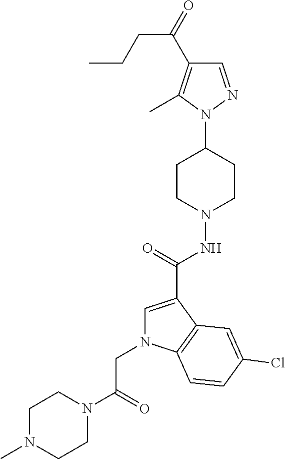 [SMILES: CCCC(=O)c1cnn(C2CCN(NC(=O)c3cn(CC(=O)N4CCN(C)CC4)c4ccc(Cl)cc34)CC2)c1C] | 0.043 |
| 5 | 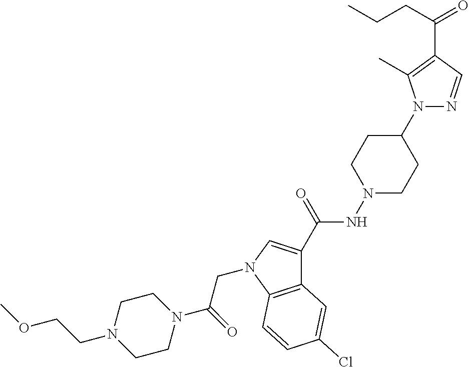 [SMILES: CCCC(=O)c1cnn(C2CCN(NC(=O)c3cn(CC(=O)N4CCN(CCOC)CC4)c4ccc(Cl)cc34)CC2)c1C] | 0.017 |
| 6 | 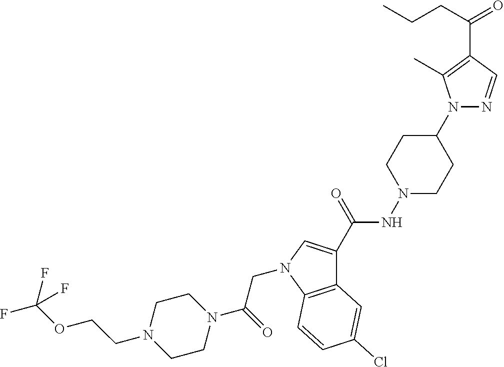 | 0.046 |

Inhibition of Platelet Aggregation In Vitro (Human Blood)

Blood is taken from healthy volunteers, using 20 ml syringes containing 2 ml of buffered sodium citrate. The blood is transferred into polypropylene tubes and centrifuged for 5 minutes (100 g) at ambient temperature (without using the brake of the centrifuge). The platelet-rich plasma (PRP) supernatant is then removed, diluted, and counted for platelets before being used in the aggregation measurements.

The platelet aggregation measurements are carried out at 37° C. in glass tubes (Chrono-Log aggregometer—Kordia). 4 μl of the test compound (solution 100 times more concentrated than the desired final concentration, in DMSO) are mixed with 392 μl of fresh PRP, and incubated for 1 minute with stirring. Then, 4 μl of a solution of ADP at 250 μM are added to the mixture. The aggregation measurements are monitored for 6 to 8 minutes, with constant stirring, by recording the variations in the optical density according to the method of G.V.R. BORN (Born Nature (1962) 194, 927).

The results are calculated using the size of the aggregation expressed as height, and expressed by the percentage inhibition.

The compounds according to the invention have IC₅₀ (platelet aggregation inhibition) values of between 0.1 and 2 μM.

Table II hereinafter indicates the results obtained for compounds 1 and 4:

TABLE II

| Com- | Inhibition of platelet aggregation |
|---|---|
| pound | in vitro (human blood) |
| No. | IC₅₀ in μM |
| 1 | 0.27 |
| 4 | 0.025 |

Inhibition of Platelet Aggregation Ex Vivo (Rat Blood)

Male rats of the Sprague-Dawley strain, weighing 250-300 g, are used in a proportion of 6 animals per batch. Each test compound is diluted in a glucose solution (5% glucose) containing 5% of cremophor and 3% of glycofurol.

The compounds according to the invention are administered by gavage or by infusion two hours before sampling.

The sample is taken on 3.8% sodium citrate (1 volume for 9 blood volumes) by abdominal aortic puncture after the animal has been anesthetized with sodium pentobarbital.

The platelet-rich plasma (PRP) is obtained by centrifugation of the blood at 300 g for 5 minutes, and the platelet aggregation measurements are carried out as described above.

The results are calculated using the area under the curve of absorbance measured for 6 minutes, and expressed by the percentage (%) inhibition.

Table III hereinafter indicates the results obtained for compounds 1 and 4:

TABLE III

| Com- | Inhibition of platelet aggregation ex vivo (rat blood) | |
|---|---|---|
| pound | Dose = 3 mg/kg and t = 2 h after administration unless specified | |
| No. | Infusion (Intravenous) | Gavage (oral) |
| 1 | 79.0 ± 5.0% (30 min) | 42.8 ± 10.7% |
| 4 | 88.0 ± 5.0% (1 mg/kg) | 72.0 ± 15.0% |

The compounds of the present invention are in particular active ingredients that are compatible with their use as medicaments and/or pharmaceutical compositions.

According to one of its aspects, the present invention relates to the use of a compound of formula (I) or of a pharmaceutically acceptable salt thereof, for preparing medicaments intended for preventing or treating any human pathological condition and/or for veterinary use. Thus, the compounds according to the invention can be used in humans or in animals (in particular in mammals, including, in a nonlimiting manner, dogs, cats, horses, bovines, sheep) for the prevention or treatment of diseases involving the P2Y12 receptor.

They are therefore indicated as inhibitors of platelet activation, of platelet aggregation and of platelet degranulation, as promoters of platelet disaggregation, and as antithrombotic agents. They are also indicated in the treatment or prophylaxis of unstable angina (pectoris), percutaneous transluminal coronary angioplasty (PCTA), myocardial infarction, perithrombolysis, thrombotic arterial complications of atherosclerosis, such as embolic or thrombotic strokes, transient ischemic events, peripheral vascular disease, myocardial infarction with or without thrombolysis, arterial complications of atherosclerosis due to surgical procedures such as angioplasty, endarterectomy, stent implantation, coronary vascular grafts and the like, thrombotic complications of surgery or mechanical damage, such as the recovery of tissues after accidental or surgical trauma, reconstructive surgery (including the skin and muscle flaps), disseminated intravascular coagulation, thrombocytopenic thrombotic purpura, hemolytic and uremic syndrome, thrombotic complications of septicemia, respiratory distress syndrome, antiphospholipid syndrome, heparin-induced thrombocytopenia and pre-eclampsia/eclampsia; or venous thromboses such as deep vein thrombosis, veno-occlusive disease, hematological conditions such as myeloproliferative disease (including thrombocythemia), sickle-cell anemia; or in the prevention of platelet activation induced mechanically in vivo, such as during cardiopulmonary bypass and extracorporeal oxygenation (prevention of microthromboembolisms), in the prevention of platelet activation induced mechanically in vitro (use in the storage of blood products, for example platelet concentrates, use during shunts such as renal dialysis and plasmapheresis), thrombosis secondary to vascular lesion/inflammation, such as angiitis, arteritis, glomerulonephritis, inflammatory bowel disease, and organ graft rejection, conditions such as migraine, Raynaud's phenomenon, conditions in which the platelets can contribute to the underlying inflammatory disease process in the vascular wall, such as the formation/progression of atheromatous plaques, stenosis/restenosis, and in other inflammatory pathological conditions, such as asthma, in which platelets and platelet-derived factors are involved in the immunological disease process.

The use of the compounds according to the invention for preventing and/or treating the diseases mentioned above, and also for preparing medicaments intended for treating these diseases, forms an integral part of the invention.

The compounds of formula (I) above, or a pharmaceutically acceptable salt thereof, can be used at daily doses of 0.01 to 100 mg per kilo body weight of the mammal to be treated, preferably at daily doses of 0.1 to 50 mg/kg. In human beings, the dose can preferably range from 0.1 to 4000 mg per day, more particularly from 0.5 to 1000 mg depending on the age of the subject to be treated or the type of treatment: prophylactic or curative.

Thus, according to another of its aspects, the present invention relates to pharmaceutical compositions containing, as active ingredient, a compound of formula (I), or a pharmaceutically acceptable salt thereof, and also one or more pharmaceutically acceptable excipients.

In the pharmaceutical compositions of the present invention for oral, sublingual, inhaled, subcutaneous, intramuscular, intravenous, topical, local, intratracheal, intranasal, transdermal, local or rectal administration, the active ingredients can be administered in unit administration forms, as a mixture with standard pharmaceutical carriers, to animals and human beings.

The appropriate unit administration forms include oral forms, such as tablets, soft or hard gel capsules, powders, granules or oral solutions or suspensions, sublingual, buccal, intratracheal, intraocular and intranasal administration forms, forms of administration by inhalation, aerosols, topical administration forms, transdermal administration forms, implants, subcutaneous, intramuscular and intravenous administration forms, and rectal administration forms.

For topical administration, the compounds according to the invention may be used in creams, ointments, gels or lotions.

By way of example, a unit administration form of a compound according to the invention in tablet form may comprise the following constituents:
Compound according to the invention: 50.0 mg
Mannitol: 223.75 mg
Sodium croscarmellose: 6.0 mg
Corn starch: 15.0 mg
Hydroxypropylmethylcellulose: 2.25 mg
Magnesium stearate: 3.0 mg

By oral administration, the dose of active ingredient administered per day can reach 0.01 to 100 mg/kg, in one or more intakes, preferentially 0.02 to 50 mg/kg.

There may be particular cases where higher or lower dosages are appropriate; such dosages do not depart from the context of the invention. According to the usual practice, the dosage that is appropriate for each patient is determined by the physician according to the mode of administration, and the weight and response of said patient.

According to another of its aspects, the present invention also relates to a method for treating the pathological conditions indicated above, which comprises the administration, to a patient, of an effective dose of a compound according to the invention, or of a pharmaceutically acceptable salt thereof.

The compounds according to the invention may also be used for preparing compositions for veterinary use.

1. A compound corresponding to formula (I): 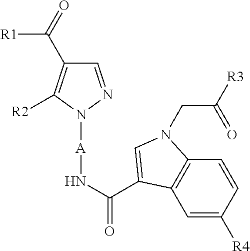
wherein
A is a divalent radical chosen from: 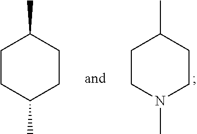
R₁ is (C₁-C₄)alkyl;
R₂ is (C₁-C₃)alkyl;
R₃ is —NR₇R₈ group,
wherein R₇ and R₈, together with the nitrogen atom to which they are bonded, form a saturated heterocycle comprising 4 to 6 ring members, which may contain another nitrogen atom,
wherein the heterocycle is substituted with at least one (C₁-C₃)alkyl which is unsubstituted or substituted with at least one of the following groups:
one, two or three halogen atoms, or
(C₁-C₃)alkoxy unsubstituted or substituted with one, two or three halogen atoms;
R₄ is halogen;
in the form of a base or of an addition salt with an acid or with a base, and also the enantiomers and diastereoisomers thereof, including the racemic mixtures thereof.

2. The compound of formula (I) as claimed in claim 1, wherein
A is 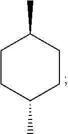
in the form of a base or of an addition salt with an acid or with a base.

3. The compound of formula (I) as claimed in claim 1, wherein
A is 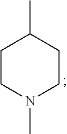
in the form of a base or of an addition salt with an acid or with a base.

4. The compound of formula (I) as claimed in claim 1, wherein R₁ is n-propyl, in the form of a base or of an addition salt with an acid or with a base.

5. The compound of formula (I) as claimed in claim 1, wherein R₃ is piperazine substituted with at least one (C₁-C₃)alkyl which is unsubstituted or substituted with at least one of the following groups:
one, two or three halogen atoms, or
(C₁-C₃)alkoxy unsubstituted or substituted with one, two or three halogen atoms;
in the form of a base or of an addition salt with an acid or with a base.

6. The compound of formula (I) as claimed in claim 1, wherein R₄ is chlorine, in the form of a base or of an addition salt with an acid or with a base.

7. The compound of formula (I) as claimed in claim 1, chosen from the following compounds:
5-Chloro-1-[2-(4-methylpiperazin-1-yl)-2-oxoethyl]-1H-indole-3-carboxylic acid [4-(4-butyryl-5-methylpyrazol-1-yl)cyclohexyl]amide
5-Chloro-1-[2-(4-methylpiperazin-1-yl)-2-oxoethyl]-1H-indole-3-carboxylic acid [4-(4-butyryl-5-methylpyrazol-1-yl)piperidin-1-yl]amide
5-Chloro-1-{2-oxo-2-[4-(3,3,3-trifluoropropyl)piperazin-1-yl]ethyl}-1H-indole-3-carboxylic acid [4-(4-butyryl-5-methylpyrazol-1-yl)cyclohexyl]amide
5-Chloro-1-{2-[4-(2-methoxyethyl)piperazin-1-yl]-2-oxoethyl}-1H-indole-3-carboxylic acid [4-(4-butyryl-5-methylpyrazol-1-yl)cyclohexyl]amide
5-Chloro-1-{2-[4-(2-methoxyethyl)piperazin-1-yl]-2-oxoethyl}-1H-indole-3-carboxylic acid [4-(4-butyryl-5-methylpyrazol-1-yl)piperidin-1-yl]amide
5-Chloro-1-{2-oxo-2-[4-(2-trifluoromethoxyethyl)piperazin-1-yl]ethyl}-1H-indole-3-carboxylic acid [4-(4-butyryl-5-methylpyrazol-1-yl)piperidin-1-yl]amide
in the form of a base or of an addition salt with an acid or with a base.

8. A process for preparing a compound of formula (I) as claimed in claim 1, wherein a compound of formula (IIB): 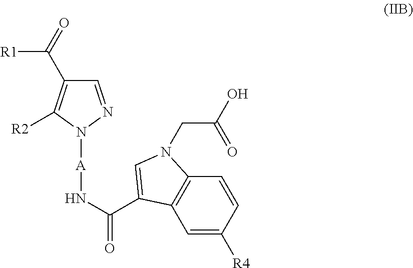
wherein A, R1, R2 and R4 are as defined for a compound of formula (I) in claim 1,
is reacted with an amine of formula (III):
HR3 (III)
wherein R3 is as defined for a compound of formula (I) in claim 1.

9. A process for preparing a compound of formula (I) as claimed in claim 1, wherein an acid, or an activated functional derivative thereof, of formula (IV-B): 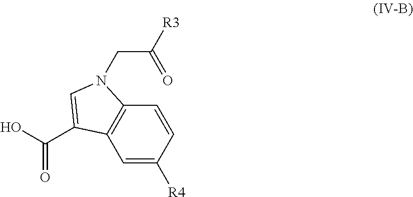
wherein R3 and R4 are as defined for a compound of formula (I) in claim 1, is reacted with a compound of formula (V): 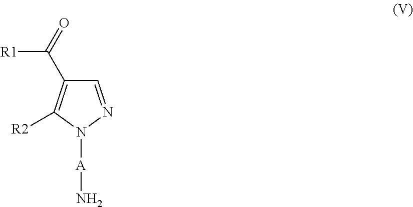
wherein A, R1 and R2 are as defined for a compound of formula (I) in claim 1.

10. A compound of formula (IIA): 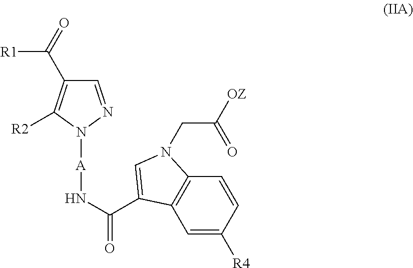
wherein
A, R1, R2 and R4 are as defined for a compound of formula (I) in claim 1; and
Z is (C₁-C₄) alkyl.

11. A compound of formula (IIB): 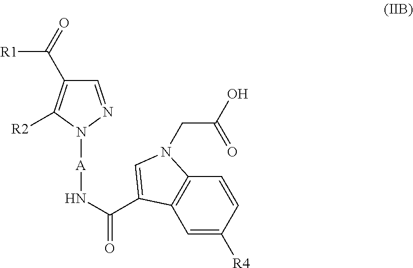
wherein A, R1, R2 and R4 are as defined for a compound of formula (I) in claim 1.

12. A harmaceutical comprising a compound of formula (I) as claimed in claim 1, or a pharmaceutically acceptable salt thereof.

13. A pharmaceutical composition comprising a compound of formula (I) as claimed in claim 1, or a pharmaceutically acceptable salt thereof, and at least one pharmaceutically acceptable excipient.

14. A method for the treatment or prophylaxis of unstable angina (pectoris), percutaneous transluminal coronary angioplasty (PCTA), myocardial infarction, perithrombolysis, thrombotic arterial complications of atherosclerosis, such as embolic or thrombotic strokes, transient ischemic events, peripheral vascular disease, myocardial infarction with or without thrombolysis, arterial complications of atherosclerosis due to surgical procedures such as angioplasty, endarterectomy, stent implantation, coronary vascular grafts and the like, thrombotic complications of surgery or mechanical damage, such as the recovery of tissues after accidental or surgical trauma, reconstructive surgery (including the skin and muscle flaps), disseminated intravascular coagulation, thrombocytopenic thrombotic purpura, hemolytic and uremic syndrome, thrombotic complications of septicemia, respiratory distress syndrome, antiphospholipid syndrome, heparin-induced thrombocytopenia and pre-eclampsia/eclampsia; or venous thromboses such as deep vein thrombosis, veno-occlusive disease, hematological conditions such as myeloproliferative disease (including thrombocythemia), sickle-cell anemia; or in the prevention of platelet activation induced mechanically in vivo, such as during cardiopulmonary bypass and extracorporeal oxygenation (prevention of microthromboembolisms), in the prevention of platelet activation induced mechanically in vitro (use in the storage of blood products, for example platelet concentrates, use during shunts such as renal dialysis and plasmapheresis), thrombosis secondary to vascular lesion/inflammation, such as angiitis, arteritis, glomerulonephritis, inflammatory bowel disease, and organ graft rejection, conditions such as migraine, Raynaud's phenomenon, conditions in which the platelets can contribute to the underlying inflammatory disease process in the vascular wall, such as the formation/progression of atheromatous plaques, stenosis/restenosis, and in other inflammatory pathological conditions, such as asthma, the method comprising the administration of an effective amount of compound of formula (I) as claimed in claim 1.
